(19) United States
(12) Patent Application Publication
(10) Pub. No.: US 2025/0170079 A1
(43) Pub. Date: May 29, 2025

(54) COMBINATIONS OF STAT-INHIBITORS AND IMMUNE CHECKPOINT INHIBITORS FOR THE TREATMENT AND/OR MANAGEMENT OF CANCER

(71) Applicant: Tvardi Therapeutics, Inc., Sugar Land, TX (US)

(72) Inventors: Imran Alibhai, Sugar Land, TX (US); Sofia de Achaval, Missouri City, TX (US); Jeno Gyuris, Lincoln, MA (US)

(21) Appl. No.: 18/842,628

(22) PCT Filed: Mar. 3, 2023
(86) PCT No.: PCT/US2023/063701

§ 371 Date: Aug. 29, 2024

Related U.S. Application Data
(60) Provisional application No. 63/268,897, filed on Mar. 4, 2022.

(51) Int. Cl. A61K 31/18; A61K 31/4196; A61K 39/00; A61P 35/00; C07K 16/28

(52) U.S. Cl. CPC A61K 31/18; A61K 31/4196; A61P 35/00; C07K 16/2818; C07K 16/2827; A61K 2039/505

(57) ABSTRACT

Provided herein are methods of treating a cancer in a subject in need thereof, comprising administering to the subject an effective amount of a STAT inhibitor and an effective amount of an immune checkpoint inhibitor (e.g., PD-1 inhibitor or PD-L1 inhibitor).

### CROSS-REFERENCE

This application is the U.S. National Stage entry of International Application No. PCT/US2023/063701, filed Mar. 3, 2023, which claims the benefit of and priority to U.S. Provisional Patent Application No. 63/268,897, filed Mar. 4, 2022, each of which is incorporated by reference in its entirety herein.

### BACKGROUND

Immune checkpoints are inhibitory pathways that help maintain self-tolerance and modulate the duration and amplitude of physiological immune responses in peripheral tissues in order to minimize collateral tissue damage. Tumors co-opt certain immune checkpoint pathways as a mechanism of immune resistance, particularly against T-cells that are specific for tumor antigens. The development of checkpoint blocking antibodies that target or are directed against, for example, programmed death 1 receptor (PD-1), can facilitate the treatment of cancers. Immunotherapies, such as anti-PD-1 or anti-PD-L1 antibody therapies, have led to improvements in treating cancers; however, there are still many patients who do not respond readily or lose responsiveness to the treatment over time. Thus, there is a need to improve cancer treatments by combining different therapies.

### SUMMARY

Provided herein, in certain embodiments, are combinations (e.g., combinations of compounds described herein, e.g., a STAT3 inhibitor and an immune checkpoint inhibitor), which can be used, for example, in methods of treating a cancer in a subject in need thereof.

In certain embodiments, provided herein are methods of treating a cancer in a subject in need thereof, comprising a) orally administering to the subject an effective amount of a compound of Formula (I):

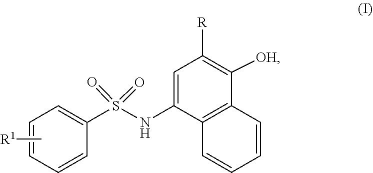

or a pharmaceutically acceptable salt thereof, wherein R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl; R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy; and b) administering to the subject an effective amount of an immune checkpoint inhibitor. In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof. In some embodiments, the compound of Formula (I) is N-(1′,2-dihydroxy-1,2′-binaphthalen-4′-yl)-4-methoxybenzenesulfonamide. In some embodiments, the immune checkpoint inhibitor is administered parenterally. In some embodiments, the immune checkpoint inhibitor is a PD-1 inhibitor or a PD-L1 inhibitor. In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab. In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072. In some embodiments, the immune checkpoint inhibitor is administered prior to administration of the compound of Formula (I). In some embodiments, the immune checkpoint inhibitor is administered concomitantly with administration of the compound of Formula (I). In some embodiments, the immune checkpoint inhibitor is administered subsequently to administration of the compound of Formula (I). In some embodiments, after three weeks or more of administration of the compound of Formula (I) and the immune checkpoint inhibitor, the subject has at least a partial remission. In some embodiments, after six weeks or more of administration of the compound of Formula (I) and the immune checkpoint inhibitor, the subject has complete remission. In some embodiments, the methods described herein reduce tumor size by at least 20% more within six weeks of administration relative to a subject administered immune checkpoint inhibitor alone.

Further provided herein, in certain embodiments, are methods of reducing the risk of recurrence of a cancer in a subject in need thereof, comprising a) orally administering to the subject an effective amount of a compound of Formula (I):

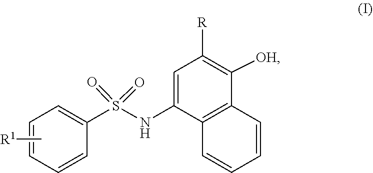

or a pharmaceutically acceptable salt thereof, wherein R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl; R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy; and b) administering to the subject an effective amount of an immune checkpoint inhibitor. In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof. In some embodiments, the compound of Formula (I) is N-(1′,2-dihydroxy-1,2′-binaphthalen-4′-yl)-4-methoxybenzenesulfonamide. In some embodiments, the immune checkpoint inhibitor is administered parenterally. In some embodiments, the immune checkpoint inhibitor is a PD-1 inhibitor or a PD-L1 inhibitor. In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab. In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072. In some embodiments, the immune checkpoint inhibitor is administered prior to administration of the compound of Formula (I). In some embodiments, the immune checkpoint inhibitor is administered concomitantly with administration of the compound of Formula (I). In some embodiments, the immune checkpoint inhibitor is administered subsequently to administration of the compound of Formula (I).

Also provided herein, in certain embodiments, are methods of preventing recurrence of a cancer in a subject in need thereof, comprising a) orally administering to the subject an effective amount of a compound of Formula (I):

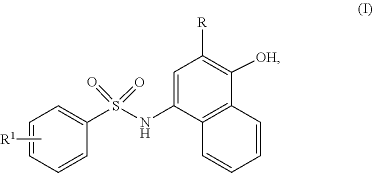

or a pharmaceutically acceptable salt thereof, wherein R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl; R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy; and b) administering to the subject an effective amount of an immune checkpoint inhibitor. In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof. In some embodiments, the compound of Formula (I) is N-(1′,2-dihydroxy-1,2′-binaphthalen-4′-yl)-4-methoxybenzenesulfonamide. In some embodiments, the immune checkpoint inhibitor is administered parenterally. In some embodiments, the immune checkpoint inhibitor is a PD-1 inhibitor or a PD-L1 inhibitor. In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab. In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072. In some embodiments, the immune checkpoint inhibitor is administered prior to administration of the compound of Formula (I). In some embodiments, the immune checkpoint inhibitor is administered concomitantly with administration of the compound of Formula (I). In some embodiments, the immune checkpoint inhibitor is administered subsequently to administration of the compound of Formula (I).

Provided herein, in certain embodiments, are methods of treating a cancer in a subject in need thereof, comprising administering to the subject a) an effective amount of a compound of Formula (II):

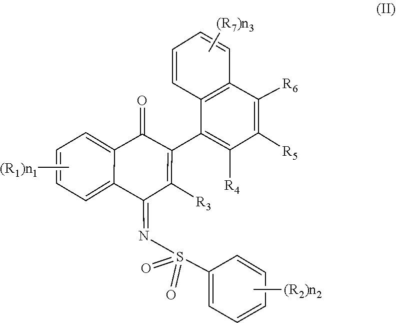

or a pharmaceutically acceptable salt thereof, wherein the variables are as defined herein, and b) an effective amount of an immune checkpoint inhibitor. In some embodiments, the compound of Formula (II) is administered orally. In some embodiments, the immune checkpoint inhibitor is administered parenterally. In some embodiments, the immune checkpoint inhibitor is a PD-1 inhibitor or a PD-L1 inhibitor. In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab. In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072. In some embodiments, the immune checkpoint inhibitor is administered prior to administration of the compound of Formula (II). In some embodiments, the immune checkpoint inhibitor is administered concomitantly with administration of the compound of Formula (II). In some embodiments, the immune checkpoint inhibitor is administered subsequently to administration of the compound of Formula (II).

Further provided herein, in certain embodiments, are methods of reducing the risk of recurrence of a cancer in a subject in need thereof, comprising administering to the subject a) an effective amount of a compound of Formula (II):

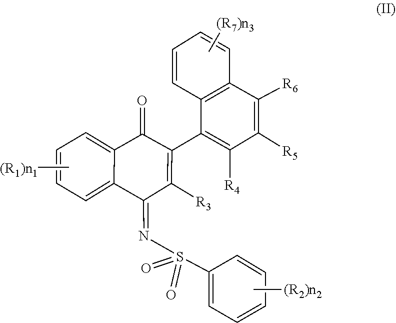

or a pharmaceutically acceptable salt thereof, wherein the variables are as defined herein, and b) an effective amount of an immune checkpoint inhibitor. In some embodiments, the compound of Formula (II) is administered orally. In some embodiments, the immune checkpoint inhibitor is administered parenterally. In some embodiments, the immune checkpoint inhibitor is a PD-1 inhibitor or a PD-L1 inhibitor. In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab. In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072. In some embodiments, the immune checkpoint inhibitor is administered prior to administration of the compound of Formula (II). In some embodiments, the immune checkpoint inhibitor is administered concomitantly with administration of the compound of Formula (II). In some embodiments, the immune checkpoint inhibitor is administered subsequently to administration of the compound of Formula (II).

Also provided herein, in certain embodiments, are methods of preventing recurrence of a cancer in a subject in need thereof, comprising administering to the subject a) an effective amount of a compound of Formula (II):

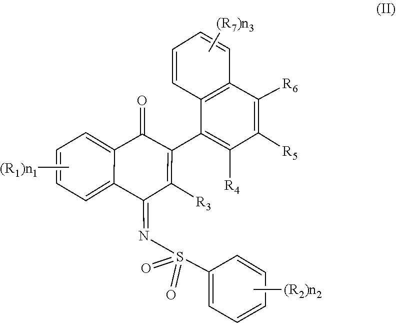

or a pharmaceutically acceptable salt thereof, wherein the variables are as defined herein, and b) an effective amount of an immune checkpoint inhibitor. In some embodiments, the compound of Formula (II) is administered orally. In some embodiments, the immune checkpoint inhibitor is administered parenterally. In some embodiments, the immune checkpoint inhibitor is a PD-1 inhibitor or a PD-L1 inhibitor. In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab. In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072. In some embodiments, the immune checkpoint inhibitor is administered prior to administration of the compound of Formula (II). In some embodiments, the immune checkpoint inhibitor is administered concomitantly with administration of the compound of Formula (II). In some embodiments, the immune checkpoint inhibitor is administered subsequently to administration of the compound of Formula (II).

Other objects and advantages will become apparent to those skilled in the art from a consideration of the ensuing Detailed Description, Examples, and Claims.

### BRIEF DESCRIPTION OF THE DRAWINGS

Various features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the in vivo efficacy results of TTI-101±anti-PD-1 in the syngeneic H22 liver cancer model through Day 21 of administration.

FIG. 2 shows the in vivo efficacy results of TTI-101±anti-PD-1 in the syngeneic H22 liver cancer model through Day 47 of administration, and Day 47.

FIG. 3 illustrates the result of a tumor rechallenge study with H22 liver cancer cells following treatment with TTI-101 and anti-PD-1 combination.

FIG. 4 illustrates the PK properties upon oral dosing of TTI-101 in SD rats.

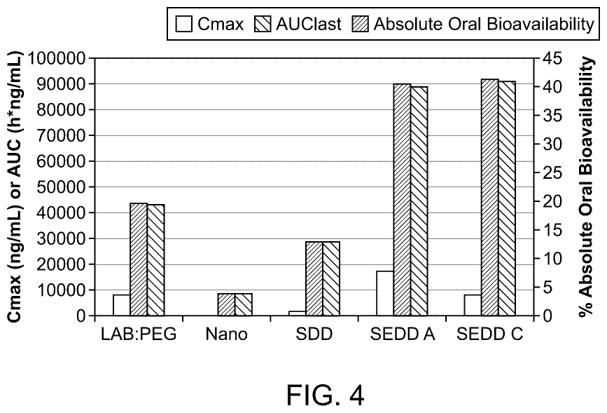

FIG. 5 illustrates percent change in tumor volume when TTI-101 is administered PO vs. IP in murine H22 liver cancer model.

### DETAILED DESCRIPTION

As generally described herein, the present disclosure provides methods of treating a cancer in a subject in need thereof or preventing recurrence of a cancer in a subject in need thereof, comprising administering to the subject an effective amount of a signal transducer and activator of transcription 3 (STAT3) inhibitor and an effective amount of an immune checkpoint inhibitor, such as PD-1 inhibitor or an PD-L1 inhibitor.

The combinations provided herein, for example, an oral administration of a STAT3 inhibitor and a parenteral administration of an immune checkpoint inhibitor, exhibit significant anti-tumor efficacies, and in some embodiments, result in complete tumor regression when compared to monotherapies alone administered for the equivalent duration and/or dosage amounts. The combinations provided herein thus provide opportunities for improving overall survival of patients, reducing the risk of developing resistance to monotherapies, and curbing adverse side effects from, for example, prolonged administration of the monotherapies. Additionally, the combinations provided herein, in some embodiments, unexpectedly protect against tumor rechallenge, thus providing opportunities to prevent or reduce risk of recurrence of cancer.

In some embodiments, the methods described herein comprise orally administering the STAT3 inhibitor. Compared to interperitoneal injection of the STAT3 inhibitor, oral administration of the STAT3 inhibitor as disclosed herein, in some embodiments, resulted in significantly greater exposure of the STAT3 inhibitor. Accordingly, the methods described herein, in some embodiments, employing oral administration of STAT3 inhibitor provide significant advantages over those employing interperitoneal administration of the STAT3 inhibitor.

### Definitions

As used herein the specification, “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit (e.g., treating, preventing, and/or ameliorating cancer in a subject, or inhibiting protein-protein interactions mediated by an SH2 domain in a subject, at a reasonable benefit/risk ratio applicable to any medical treatment) in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

The term “inhibitor” as used herein, for example with respect to STAT3 inhibitors, refers to one or more molecules that interfere at least in part with the activity of Stat3 to perform one or more activities, including the ability of Stat3 to bind to a molecule and/or the ability to be phosphorylated.

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The term “alkyl” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄) alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy (E)-but-2-enyl, 2-methy (Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc and Rd is independently hydrogen, alkyl, cycloalkyl, heterocycle, aryl, or said Rb and Rc, together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted.

The term “cycloalkyl” refers to a fully-saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl, or naphthyl. Where containing two or more aromatic rings (bicyclic, etc.), the aromatic rings of the aryl group may be joined at a single point (e.g., biphenyl), or fused (e.g., naphthyl, phenanthrenyl, and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “carbocycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms, and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl], or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORa, SRa, S(═O)Re, S(═O)₂Re, P(═O)₂Re, S(═O)₂ORe, P(═O)₂ORe, NRbRc, NRbS(═O)₂Re, NRbP(═O)₂Re, S(═O)₂NRbRc, P(═O)₂NRbRc, C(═O)ORd, C(═O)Ra, C(═O)NRbRc, OC(═O)Ra, OC(═O)NRbRc, NRbC(═O)ORe, NRdC(═O)NRbRc, NRdS(═O)₂NRbRc, NRdP(═O)₂NRbRc, NRbC(═O)Ra, or NRbP(═O)₂Re, wherein each occurrence of Ra is independently hydrogen, alkyl, cycloalkyl, alkenyl, cycloalkenyl, alkynyl, heterocycle, or aryl; each occurrence of Rb, Rc, and Rd is independently hydrogen, alkyl, cycloalkyl, heterocycle, aryl, or said Rb and Rc together with the N to which they are bonded optionally form a heterocycle; and each occurrence of Re is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “alkylamino” refers to a group having the structure —NHR′, wherein R′ is hydrogen, alkyl or substituted alkyl, or cycloalkyl or substituted cyclolalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, or heterocylyl or substituted heterocyclyl, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In some embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine, or iodine.

Unless otherwise indicated, any heteroatom with unsatisfied valences is assumed to have hydrogen atoms sufficient to satisfy the valences.

Compounds of the present disclosure, and salts or solvates thereof, may exist in their tautomeric form (for example, as an amide or imino ether). All such tautomeric forms are contemplated herein as part of the present disclosure.

All stereoisomers of the present compounds (for example, those which may exist due to asymmetric carbons on various substituents), including enantiomeric forms and diastereomeric forms, are contemplated within the scope of this disclosure. Individual stereoisomers of the compounds of the disclosure may, for example, be substantially free of other isomers (e.g., as a pure or substantially pure optical isomer having a specified activity), or may be admixed, for example, as racemates or with all other, or other selected, stereoisomers. The chiral centers of the present disclosure may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present disclosure are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds of the present disclosure are also contemplated herein as part of the present disclosure.

All configurational isomers of the compounds of the present disclosure are contemplated, either in admixture or in pure or substantially pure form. The definition of compounds of the present disclosure embraces both cis (Z) and trans (E) alkene isomers, as well as cis and trans isomers of cyclic hydrocarbon or heterocyclic rings.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in “Organic Chemistry,” Thomas Sorrell, University Science Books, Sausalito (1999), the entire contents of which are incorporated herein by reference.

Certain compounds of the present disclosure may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

Isomeric mixtures containing any of a variety of isomer ratios may be utilized in accordance with the present disclosure. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present disclosure. Those of ordinary skill in the art will readily appreciate that analogous ratios are contemplated for more complex isomer mixtures.

The present disclosure also includes isotopically-labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorous, sulfur, fluorine, and chlorine, such as 2H, 3H, 13C, 11C, 14C, 15N, 18O, 17O, 31P, 32P, 35S, 18F, and 36Cl, respectively. Compounds of the present disclosure, or an enantiomer, diastereomer, tautomer, or pharmaceutically acceptable salt or solvate thereof, which contain the aforementioned isotopes and/or other isotopes of other atoms are within the scope of this disclosure. Certain isotopically-labeled compounds of the present disclosure, for example those into which radioactive isotopes such as 3H and 14C are incorporated, are useful in drug and/or substrate tissue distribution assays. Tritiated, i.e., 3H, and carbon-14, i.e., 14C, isotopes are particularly preferred for their ease of preparation and detectability. Further, substitution with heavier isotopes such as deuterium, i.e., 2H, can afford certain therapeutic advantages resulting from greater metabolic stability, for example increased in vivo half-life or reduced dosage requirements and hence, may be preferred in some circumstances. Isotopically labeled compounds can generally be prepared by carrying out the procedures disclosed in the Schemes and/or in the Examples below, by substituting a readily-available isotopically labeled reagent for a non-isotopically labeled reagent.

If, for instance, a particular enantiomer of a compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this disclosure, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic, substituents of organic compounds. For purposes of this disclosure, heteroatoms such as nitrogen may have hydrogen substituents and/or any permissible substituents of organic compounds described herein which satisfy the valencies of the heteroatoms. Furthermore, this disclosure is not intended to be limited in any manner by the permissible substituents of organic compounds. Combinations of substituents and variables envisioned by this disclosure are preferably those that result in the formation of stable compounds useful in the treatment, for example, of infectious diseases or proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

### Methods of Treatment

Disclosed herein, in certain embodiments, are methods of treating a cancer in a subject in need thereof, comprising a) orally administering to the subject an effective amount of a compound of Formula (I):

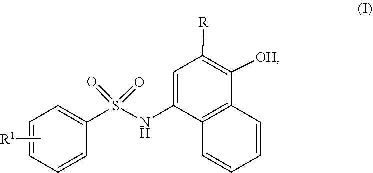

or a pharmaceutically acceptable salt thereof, wherein R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl; R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy; and b) administering to the subject an effective amount of an immune checkpoint inhibitor.

In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

In some embodiments, the compound of Formula (I) is N-(1′,2-dihydroxy-1,2′-binaphthalen-4′-yl)-4-methoxybenzenesulfonamide.

In certain embodiments, the immune checkpoint inhibitor is administered parenterally.

In certain embodiments, the immune checkpoint inhibitor is a PD-1 inhibitor or a PD-L1 inhibitor.

In some embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab.

In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

In some embodiments, the immune checkpoint inhibitor is administered prior to administration of the compound of Formula (I). In some embodiments, the immune checkpoint inhibitor is administered concomitantly with administration of the compound of Formula (I). In some embodiments, the immune checkpoint inhibitor is administered subsequently to administration of the compound of Formula (I). In some embodiments, after three weeks or more of administration of the compound of Formula (I) and the immune checkpoint inhibitor, the subject has at least a partial remission. In some embodiments, after six weeks or more of administration of the compound of Formula (I) and the immune checkpoint inhibitor, the subject has complete remission. In some embodiments, the methods described herein reduce tumor size by at least 20% more within six weeks of administration relative to a subject administered immune checkpoint inhibitor alone.

Disclosed herein, in certain embodiments, are methods of reducing the risk of recurrence of a cancer in a subject in need thereof, comprising a) orally administering to the subject an effective amount of a compound of Formula (I):

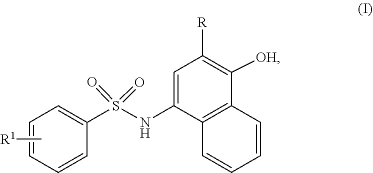

or a pharmaceutically acceptable salt thereof, wherein R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl; R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy; and b) administering to the subject an effective amount of an immune checkpoint inhibitor.

In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

In some embodiments, the compound of Formula (I) is N-(1′,2-dihydroxy-1,2′-binaphthalen-4′-yl)-4-methoxybenzenesulfonamide.

In certain embodiments, the immune checkpoint inhibitor is administered parenterally.

In certain embodiments, the immune checkpoint inhibitor is a PD-1 inhibitor or a PD-L1 inhibitor.

In certain embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab.

In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

In some embodiments, the immune checkpoint inhibitor is administered prior to administration of the compound of Formula (I). In some embodiments, the immune checkpoint inhibitor is administered concomitantly with administration of the compound of Formula (I). In some embodiments, the immune checkpoint inhibitor is administered subsequently to administration of the compound of Formula (I).

Disclosed herein, in certain embodiments, are methods of preventing recurrence of a cancer in a subject in need thereof, comprising a) orally administering to the subject an effective amount of a compound of Formula (I):

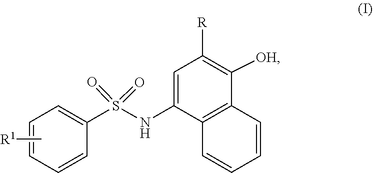

or a pharmaceutically acceptable salt thereof, wherein R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl; R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy; and b) administering to the subject an effective amount of an immune checkpoint inhibitor.

In some embodiments, the compound of Formula (I) is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

In some embodiments, the compound of Formula (I) is N-(1′,2-dihydroxy-1,2′-binaphthalen-4′-yl)-4-methoxybenzenesulfonamide.

In certain embodiments, the immune checkpoint inhibitor is administered parenterally.

In certain embodiments, the immune checkpoint inhibitor is a PD-1 inhibitor or a PD-L1 inhibitor.

Disclosed herein, in certain embodiments, are methods of treating a cancer in a subject in need thereof, comprising administering to the subject a) an effective amount of a compound of Formula (II):

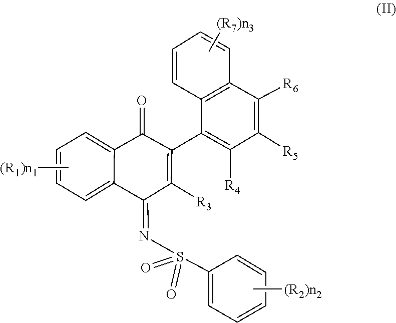

or a pharmaceutically acceptable salt thereof, wherein the variables are as defined herein, and b) an effective amount of an immune checkpoint inhibitor.

In certain embodiments, the compound of Formula (II) or a pharmaceutically acceptable salt thereof is orally administered.

In certain embodiments, the immune checkpoint inhibitor is administered parenterally.

In certain embodiments, the immune checkpoint inhibitor is a PD-1 inhibitor or a PD-L1 inhibitor.

In certain embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab.

In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

In some embodiments, the immune checkpoint inhibitor is administered prior to administration of the compound of Formula (II). In some embodiments, the immune checkpoint inhibitor is administered concomitantly with administration of the compound of Formula (II). In some embodiments, the immune checkpoint inhibitor is administered subsequently to administration of the compound of Formula (II).

Disclosed herein, in certain embodiments, are methods of reducing the risk of recurrence of a cancer in a subject in need thereof, comprising administering to the subject a) an effective amount of a compound of Formula (II):

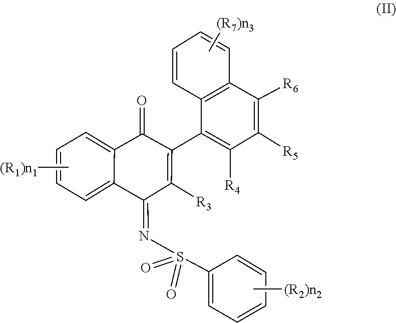

or a pharmaceutically acceptable salt thereof, wherein the variables are as defined herein, and b) an effective amount of an immune checkpoint inhibitor.

In certain embodiments, the compound of Formula (II) or a pharmaceutically acceptable salt thereof is orally administered.

In certain embodiments, the immune checkpoint inhibitor is administered parenterally.

In certain embodiments, the immune checkpoint inhibitor is a PD-1 inhibitor or a PD-L1 inhibitor.

In certain embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab.

In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

In some embodiments, the immune checkpoint inhibitor is administered prior to administration of the compound of Formula (II). In some embodiments, the immune checkpoint inhibitor is administered concomitantly with administration of the compound of Formula (II). In some embodiments, the immune checkpoint inhibitor is administered subsequently to administration of the compound of Formula (II).

Disclosed herein, in certain embodiments, are methods of preventing recurrence of a cancer in a subject in need thereof, comprising administering to the subject a) an effective amount of a compound of Formula (II):

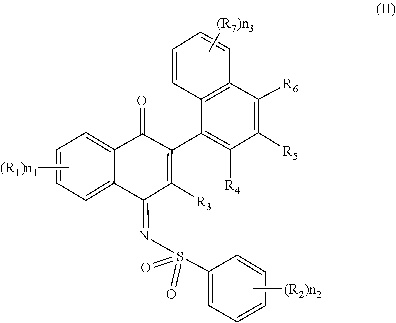

or a pharmaceutically acceptable salt thereof, wherein the variables are as defined herein, and b) an effective amount of an immune checkpoint inhibitor.

In certain embodiments, the compound of Formula (II) or a pharmaceutically acceptable salt thereof is orally administered.

In certain embodiments, the immune checkpoint inhibitor is administered parenterally.

In certain embodiments, the immune checkpoint inhibitor is a PD-1 inhibitor or a PD-L1 inhibitor.

In certain embodiments, the PD-1 inhibitor is an anti-PD-1 antibody. In some embodiments, the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717. In some embodiments, the anti-PD-1 antibody is pembrolizumab.

In some embodiments, the PD-L1 inhibitor is an anti-PD-L1 antibody. In some embodiments, the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

In some embodiments, the immune checkpoint inhibitor is administered prior to administration of the compound of Formula (II). In some embodiments, the immune checkpoint inhibitor is administered concomitantly with administration of the compound of Formula (II). In some embodiments, the immune checkpoint inhibitor is administered subsequently to administration of the compound of Formula (II).

### Targeting STAT Proteins

STAT proteins, of which there are seven (1, 2, 3, 4, 5A, 5B and 6), transmit peptide hormone signals from the cell surface to the nucleus. STAT inhibitors, as used herein, are compounds that target STAT proteins.

In some embodiments, a STAT3 inhibitor, as used herein, refers to one or more molecules that interfere at least in part with the activity of STAT3 to perform one or more activities, including the ability of STAT3 to bind to a molecule and/or the ability to be phosphorylated. In some embodiments, the STAT3 inhibitor interacts with the Stat3 SH2 domain, competitively inhibits recombinant Stat3 binding to its immobilized pY-peptide ligand, and/or inhibits IL-6-mediated tyrosine phosphorylation of Stat3, for example. In some embodiments, the STAT3 inhibitor fulfills the criteria of interaction analysis (CIA): 1) global minimum energy score≤−30; 2) formation of a salt-bridge and/or H-bond network within the pY-residue binding site of Stat3; and/or 3) formation of a H-bond with or blocking access to the amide hydrogen of E638 of Stat3, for example. In some embodiments, the STAT3 inhibitor interacts with a hydrophobic binding pocket with the Stat3 SH2 domain. In some embodiments, the STAT3 inhibitor inhibits the binding of Stat3 to its cognate phosphopeptide ligand. In some embodiments, the STAT3 inhibitor inhibits cytokine-mediated Stat3 phosphorylation within cells. In some embodiments, the STAT3 inhibitor inhibits nuclear translocation of Stat3 within cells.

In some embodiments, the STAT inhibitors described herein are STAT1 inhibitors. Studies in Stat1-deficient mice (Meraz et al., 1996; Durbin et al., 1996; Ryan et al., 1998) support an essential role for Stat1 in innate immunity, notably against viral pathogens. In addition, Stat1 is a potent inhibitor of growth and promoter of apoptosis (Bromberg and Darnell, 2000). Also, because tumors from carcinogen-treated wild-type animals grow more rapidly when transplanted into the Stat1-deficient animals than they do in a wild-type host, Stat1 contributes to tumor surveillance (Kaplan et al., 1998).

In some embodiments, the STAT inhibitors described herein are STAT5 inhibitors. STAT5 inhibitors refer to one or more molecules that interfere at least in part with the activity of STAT5 to perform one or more activities, including the ability of STAT5 to bind to a molecule and/or the ability to be phosphorylated.

In some embodiments, the STAT inhibitor (e.g., a STAT3 inhibitor) is a compound of Formula (I):

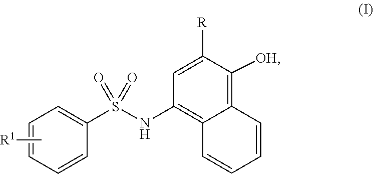

wherein R is selected from the group consisting of hydrogen, phenylsulfanyl, hydroxynaphthalenyl, quinolinylsulfanyl, triazolyl sulfanyl, and benzothiazolylsulfanyl,
wherein R¹ is selected from the group consisting of hydrogen, methyl, chloro, bromo, methoxy, ethoxy, tert-butyl, nitro, methyl ester, acetamide, 1,4-dioxine, fluoro, trifluoro methoxy, acetyl, trifluoro methyl, propyl, cyclohexene, methoxyphenoxy, chlorophenoxy, tolyloxy, and phenoxy.

In some embodiments, the STAT inhibitor is N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof. In some embodiments, the STAT inhibitor is N-(1′,2-dihydroxy-1,2′-binaphthalen-4′-yl)-4-methoxybenzenesulfonamide (also referred to herein as TTI-101).

In some embodiments, the STAT inhibitor is a compound of Formula (II):

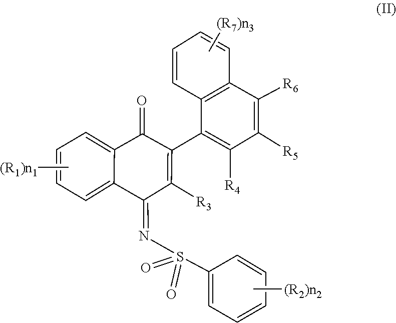

or a pharmaceutically acceptable salt thereof, wherein each occurrence of R¹ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₁ is 0, 1, 2, 3, or 4;
each occurrence of R₂ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, cycloalkenyl, optionally substituted aryl, optionally substituted aryloxyl, or optionally substituted heterocycle;
n₂ is 0, 1, 2, 3, 4, or 5;
R₃ is hydrogen, halogen, cyano, nitro, CF₃, OCF₃, ORa, SRa, OC(═O)Ra, alkyl, alkenyl, cycloalkyl, or optionally substituted aryl or heteroaryl;
R₄ is hydrogen, halogen, cyano, nitro, CF₃, OCF₃, ORa, SRa, NRbRc, OC(═O)Ra, alkyl, alkenyl, or cycloalkyl;
each occurrence of R₅, R₆, and R₇ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₃ is 0, 1, 2, 3, or 4; and
each occurrence of Ra, Rb, and Rc is independently hydrogen, alkyl, cycloalkyl, alkenyl, cycloalkenyl, alkynyl, heterocycle, or aryl; or said Rb and Rc together with the nitrogen atom to which they are bonded optionally form a heterocycle comprising 1-4 heteroatoms.

In some embodiments of Formula (II), each occurrence of R₁ is independently hydrogen, halogen, cyano, nitro, CF₃, OCF₃, ORa, or SRa. In some embodiments of Formula (II), each occurrence of R¹ is independently C(═O)Ra, OC(═O)Ra, C(═O)ORa, NRaRb, NRbC(═O)Ra, C(═O)NRbRc, NRbC(═O)ORa, OC(═O)NRbRc, or NRaC(═O)NRbRc. In some embodiments of Formula (II), each occurrence of R¹ is independently alkyl, alkenyl, cycloalkyl, optionally substituted aryl, or optionally substituted heterocycle. In some embodiments of Formula (II), R₁ is hydrogen.

In some embodiments of Formula (II), n₁ is 0, 1, or 2. In some embodiments of Formula (II), n₁ is 1. In some embodiments of Formula (II), n₁ is 0.

In some embodiments of Formula (II), each occurrence of R₂ is independently hydrogen, halogen, cyano, nitro, CF₃, OCF₃, ORa, or SRa. In some embodiments of Formula (II), each occurrence of R₂ is independently C(═O)Ra, OC(═O)Ra, C(═O)ORa, NRaRb, NRbC(═O)Ra, C(═O)NRbRc, NRbC(═O)ORa, OC(═O)NRbRc, or NRaC(═O)NRbRc. In some embodiments of Formula (II), each occurrence of R₂ is independently alkyl, alkenyl, cycloalkyl, optionally substituted aryl, or optionally substituted heterocycle. In some embodiments of Formula (II), R₂ is hydrogen.

In some embodiments of Formula (II), n₂ is 0, 1, or 2. In some embodiments of Formula (II), n₂ is 1. In some embodiments of Formula (II), n₂ is 0.

In some embodiments of Formula (II), R₃ is hydrogen, halogen, cyano, nitro, or CF₃. In some embodiments of Formula (II), R₃ is OCF₃, ORa, SRa, or OC(═O)Ra. In some embodiments of Formula (II), R₃ is alkyl, alkenyl, or cycloalkyl. In some embodiments of Formula (II), R₃ is hydrogen.

In some embodiments of Formula (II), R₄ is hydrogen, halogen, cyano, nitro, or ORa. In some embodiments of Formula (II), R₄ is OCF₃, SRa, or OC(═O)Ra. In some embodiments of Formula (II), R₄ is alkyl, alkenyl, or cycloalkyl. In some embodiments of Formula (II), R₄ is OH. In some embodiments of Formula (II), R₄ is OMe.

In some embodiments of Formula (II), R₅, R₆, and R₇ are each independently selected from the group consisting of hydrogen, halogen, cyano, nitro, and CF₃. In some embodiments of Formula (II), R₅, R₆, and R₇ are each independently selected from the group consisting of OCF₃, ORa, and SRa. In some embodiments of Formula (II), R₅, R₆, and R₇ are each independently selected from the group consisting of OCF₃ and ORa. In some embodiments of Formula (II), R₅, R₆, and R₇ are each independently selected from the group consisting of C(═O)Ra, OC(═O)Ra, C(═O)ORa, NRaRb, NRbC(═O)Ra, C(═O)NRbRc, NRbC(═O)ORa, OC(═O)NRbRc, and NRaC(═O)NRbRc. In some embodiments of Formula (II), R₅, R₆, and R₇ are each independently selected from the group consisting of alkyl, alkenyl, cycloalkyl, optionally substituted aryl, and optionally substituted heterocycle. In some embodiments of Formula (II), each occurrence of R₅, R₆, and R₇ is hydrogen.

In some embodiments of Formula (II), n₃ is 0, 1, or 2. In some embodiments of Formula (II), n₃ is 1. In some embodiments of Formula (II), n₃ is 0.

In some embodiments of Formula (II), each occurrence of Ra is independently hydrogen, alkyl, heterocycle, or aryl. In some embodiments of Formula (II), each occurrence of Ra is independently hydrogen or alkyl. In some embodiments of Formula (II), each occurrence of Rb and Rc is independently hydrogen, alkyl, heterocycle, or aryl. In some embodiments of Formula (II), each occurrence of Rb and Rc is independently hydrogen or alkyl. In some embodiments of Formula (II), Rb and Rc together with the nitrogen atom to which they are bonded optionally form a heterocycle comprising 1-4 heteroatoms each selected from the group consisting of N, O, and S.

In some embodiments, the compound of Formula (II) is a compound of Formula (III):

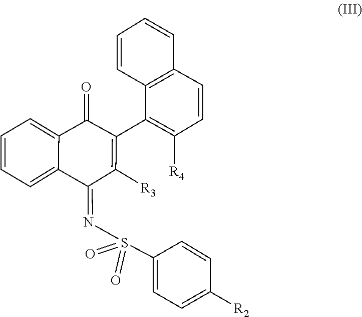

or a pharmaceutically acceptable salt thereof.

In some embodiments of Formula (III), R₂ is H, OH, alkyl, alkoxy, halogen, NRbRc, CF₃, OCF₃, or CN. In some embodiments of Formula (III), R₂ is NH₂, OH, OMe, OEt, OCH₂CH₂CH₃, or OCH(CH₃)₂. In some embodiments of Formula (III), R₂ is selected from the group consisting of hydrogen, methyl, ethyl, propyl, tert-butyl, F, Cl, Br, CF₃, nitro, methoxy, ethoxy, OCF₃, —C(═O)Me, —C(═O)OMe, —NHC(═O)Me, 1,4-dioxanyl, cyclohexanyl, cyclohexenyl, phenoxy, 2-methoxyphenoxy, 3-methoxyphenoxy, 4-methoxyphenoxy, 2-chlorophenoxy, 3-chlorophenoxy, 4-chlorophenoxy, 2-methylphenoxy, 3-methylphenoxy, and 4-methylphenoxy. In some embodiments of Formula (III), R₂ is OMe.

In some embodiments of Formula (III), R₃ is H, OH, alkyl, alkoxy, or halogen. In some embodiments of Formula (III), R₃ is H.

In some embodiments of Formula (III), R₄ is H, alkyl, OH, NH₂, alkoxy, halogen, CF₃, or CN. In some embodiments of Formula (III), R₄ is H, OH, or alkoxy. In some embodiments of Formula (III), R₄ is OH. In some embodiments of Formula (III), R₄ is OMe.

In some embodiments, the compound of Formula (III) is a compound of Formula (IV):

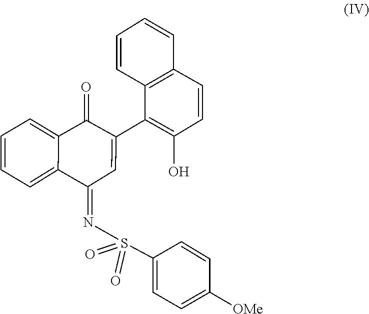 [SMILES: COc1ccc(S(=O)(=O)/N=C2\C=C(c3c(O)ccc4ccccc34)C(=O)c3ccccc32)cc1]

or a pharmaceutically acceptable salt thereof.

In some embodiments, the compound of Formula (II) is a compound selected from Examples of compounds shown in Table A, or a pharmaceutically acceptable salt thereof.

TABLE A
Selected compounds of Formula (II), where n₁, n₂, and n₃ are independently 1 or 2.

| Ex- | | | | | | | |
|---|---|---|---|---|---|---|---|
| ample | R₁ | R₂ | R₃ | R₄ | R₅ | R₆ | R₇ |
| 100 | H | Cl | H | H | H | F | F |
| 102 | F | H | F | H | F | OH | Cl |
| 103 | Cl | 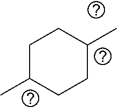 | Cl | OH | Cl | OH | NO₂ [symbol] |
| 104 | CN | OH | CN | OH | Br | OMe | OCF₃ |
| 105 | NO₂ | OMe | CF₃ | OH | NO₂ | NH₂ | SH |
| 106 | CF₃ | OEt | Me | OMe | OCF₃ | NH₂ | SH |
| 107 | OCF₃ | OPr | Et | NH₃ | OCF₃ | SCH₃ | OH |
| 108 | OH | OBu | Pr | NH₃ | SH | COOH | CONH₂ |
| 109 | OH | NH₂ | Bu | NH₃ | SH | COOH | CONH₂ |
| 110 | SH | SH | Cyclo- | SH | OH | Ph | OH |
| | | | propyl | | | | |
| 111 | COOH | Me | H | SCH₃ | OH | pyridinyl | OH |
| 112 | COOMe | CONH₂ | H | COOH | CONH₂ | Ph | OH |
| 113 | CONH₂ | NH(C═O)Me | —CH═CH₂ | COOH | CONH₂ | Me | H |
| 114 | CONMe₂ | cyclopropyl | Ph | OH | CONH₂ | Et | H |
| 115 | NH(C═O)Me | Ph | pyridinyl | OH | NH(C═O)Me | Pr | Me |
| 116 | Me | 3-fluorophenyl | Me | OH | Me | cyclobutyl | Et |
| 117 | Ph | 4-pyridinyl | H | NHMe | Et | CF₃ | cyclopropyl |
| 118 | 4-chlorophenyl | NO₂ | H | NHMe | cyclobutyl | OCF₃ | NO₂ |
| 119 | 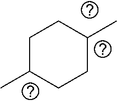 | CF₃ | H | NMe₂ | 4-chlorophenyl | SH | pyridinyl |
| 120 | cyclobutyl | OH | H | 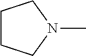 | Ph | Ph | 4-pyridinyl |
| 121 | 4-pyridinyl | SH | H | 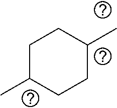 | OEt | 4-chlorophenyl | CONMe₂ |
| 122 | OEt | Me | Me | Me | OPr | OPr | NH(C═O)Me |
| 123 | OPr | Et | Ph | Me | 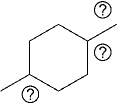 | 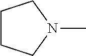 | 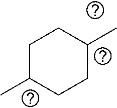 |
| 124 | OBu | Pr | Ph | Et | 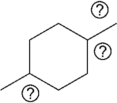 | 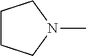 | 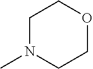 |

[symbol]

indicates data missing or illegible when filed

In some embodiments, the compound of Formula (III) is a compound selected from Examples of compounds shown in Table B, or a pharmaceutically acceptable salt thereof.

TABLE B
Selected compounds of Formula (III).

| Example No. | R₂ | R₃ | R₄ |
|---|---|---|---|
| 125 | Cl | H | H |
| 126 | H | F | OH |
| 127 | 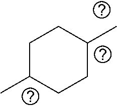 | Cl | OH |
| 128 | OH | H | OH |
| 129 | OMe | CF₃ | OH |
| 130 | OEt | Me | OMe |
| 131 | OPr | Et | NH₂ |
| 132 | OBu [symbol] | Pr | NH₂ |
| 133 | NH₂ | Bu | NH₂ |
| 134 | SH | cyclopropyl | SH |
| 135 | Me | H | SCH₃ |
| 136 | CONH₂ | H | COOH |
| 137 | NH(C═O)Me | —CH═CH₂ | COOH |
| 138 | cyclopropyl | Ph | OH |
| 139 | Ph | pyridinyl | OH |
| 140 | 3-fluorophenyl | Me | OH |
| 141 | 4-pyridinyl | H | NHMe |
| 142 | NO₂ | H | NHMe |
| 143 | CF₃ | H | NMe₂ |
| 144 | OH | H | 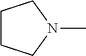 |
| 145 | SH | H | 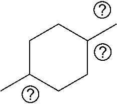 |
| 146 | Me | Me | Me |
| 147 | Et | Ph | Me |
| 148 | Pr | Ph | Et |

[symbol]

indicates data missing or illegible when filed

### Immune Checkpoint Inhibitors

Immune checkpoint inhibitors block proteins called checkpoints that are made by some types of immune system cells, such as T cells, and some cancer cells. These checkpoints help keep immune responses from being too strong and sometimes can keep T cells from killing cancer cells. When these checkpoints are blocked, T cells can kill cancer cells better.

### PD-1 Inhibitors and PD-L1 Inhibitors

Disclosed herein, in certain embodiments, are methods of treating a cancer in a subject in need thereof and preventing recurrence of a cancer in a subject in need thereof, comprising administering to the subject an effective amount of a signal transducer and activator of transcription 3 (STAT3) inhibitor and an effective amount of an immune checkpoint inhibitor, such as PD-1 inhibitor or an PD-L1 inhibitor. In some embodiments, the PD-1 inhibitor is an anti-PD-1 antibody. In some embodiments, the PD-L1 inhibitor is an anti-PD-L1 antibody.

Antibody therapies are antibody proteins produced by the immune system and that bind to a target antigen on the surface of a cell. Antibodies are typically encoded by an immunoglobulin gene or genes, or fragments thereof. In normal physiology antibodies are used by the immune system to fight pathogens. Each antibody is specific to one or a few proteins, and those that bind to cancer antigens are used, e.g., for the treatment of cancer. Antibodies are capable of specifically binding an antigen or epitope. (Fundamental Immunology, 3rd Edition, W.e., Paul, ed., Raven Press, N.Y. (1993). Specific binding occurs to the corresponding antigen or epitope even in the presence of a heterogeneous population of proteins and other biologies. Specific binding of an antibody indicates that it binds to its target antigen or epitope with an affinity that is substantially greater than binding to irrelevant antigens. The relative difference in affinity is often at least 25% greater, more often at least 50% greater, most often at least 100% greater. The relative difference can be at least 2-fold, at least 5-fold, at least 10-fold, at least 25-fold, at least 50-fold, at least 100-fold, or at least 1000-fold, for example.

Exemplary types of antibodies include without limitation human, humanized, chimeric, monoclonal, polyclonal, single chain, antibody binding fragments, and diabodies. Once bound to a cancer antigen, antibodies can induce antibody-dependent cell-mediated cytotoxicity, activate the complement system, prevent a receptor interacting with its ligand or deliver a payload of chemotherapy or radiation, all of which can lead to cell death.

In some embodiments, anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717.

In some embodiments, anti-PD-L1 antibody is anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

### Diseases and Disorders

Signal transducer and activator of transcription 3 (STAT3) is central in regulating the anti-tumor immune response. STAT3 is broadly hyperactivated both in cancer and non-cancerous cells within the tumor ecosystem and plays important roles in inhibiting the expression of crucial immune activation regulators and promoting the production of immunosuppressive factors. Methods provided herein are contemplated as being useful for the treatment of a cancer, including for example, solid tumors, soft tissue tumors, and metastases thereof.

In some embodiments, a cancer treated according to a method provided herein is a liver cancer, lung cancer, head and neck cancer, breast cancer, skin cancer, kidney cancer, testicular cancer, colon cancer, rectal cancer, gastric cancer, metastatic melanoma, prostate cancer, ovarian cancer, cervical cancer, bone cancer, spleen cancer, gall bladder cancer, brain cancer, pancreatic cancer, stomach cancer, anal cancer, prostate cancer, multiple myeloma, post-transplant lymphoproliferative disease, restenosis, myelodysplastic syndrome, leukemia, lymphoma, or acute myelogenous leukemia. In some embodiments, a cancer treated according to a method provided herein is a liver cancer, lung cancer, hepatocellular carcinoma, head and neck squamous cell carcinoma, non-small cell lung cancer, or estrogen receptor-positive breast cancer.

In some embodiments, a cancer treated according to a method provided herein is head and neck cancer, lung cancer, liver cancer, breast cancer, ovarian cancer, colon cancer, multiple myeloma, leukemia, or pancreatic cancer. In some embodiments, the leukemia is acute myelogenous leukemia.

### Combinations

The phrase, “in combination with,” and the terms “co-administration,” “co-administering,” or “co-providing”, as used herein in the context of the administration of a compound described herein or a therapy described herein, means that two (or more) different compounds or therapies are delivered to the subject during the course of the subject's affliction with the disease or disorder (e.g., a disease or disorder as described herein, e.g., cancer), e.g., two (or more) different compounds or therapies are delivered to the subject after the subject has been diagnosed with the disease or disorder (e.g., a disease or disorder as described herein, e.g., cancer) and before the disease or disorder has been cured or eliminated or treatment has ceased for other reasons.

In some embodiments, the delivery of one compound or therapy is still occurring when the delivery of the second begins, so that there is overlap in terms of administration. This is sometimes referred to herein as “simultaneous” or “concurrent delivery.” In other embodiments, the delivery of one compound or therapy ends before the delivery of the other compound or therapy begins. In some embodiments of either case, the treatment (e.g., administration of compound, composition, or therapy) is more effective because of combined administration. For example, the second compound or therapy is more effective, e.g., an equivalent effect is seen with less of the second compound or therapy, or the second compound or therapy reduces symptoms to a greater extent, than would be seen if the second compound or therapy were administered in the absence of the first compound or therapy, or the analogous situation is seen with the first compound or therapy. In some embodiments, delivery is such that the reduction in a symptom, or other parameter related to the disorder is greater than what would be observed with one compound or therapy delivered in the absence of the other. The effect of the two compounds or therapies can be partially additive, wholly additive, or greater than additive (e.g., synergistic). The delivery can be such that the first compound or therapy delivered is still detectable when the second is delivered.

In some embodiments, the first compound or therapy and second compound or therapy can be administered simultaneously (e.g., at the same time), in the same or in separate compositions, or sequentially. Sequential administration refers to administration of one compound or therapy before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, compound or therapy. The order of administration of the first and secondary compound or therapy can also be reversed.

The combinations described herein can be a first line treatment for cancer, i.e., it is used in a patient who has not been previously administered another drug intended to treat the cancer; a second line treatment for the cancer, i.e., it is used in a subject in need thereof who has been previously administered another drug intended to treat the cancer; a third or fourth treatment for the cancer, i.e., it is used in a subject who has been previously administered two or three other drugs intended to treat the cancer.

### Administration and Dosages

The combinations of this disclosure may be administered orally, parenterally, topically, rectally, or via an implanted reservoir, preferably by oral administration (e.g., for STAT3 inhibitor) or administration by injection (e.g., for anti-PD-1). In some cases, the pH of a composition (e.g., pharmaceutical composition) comprising a STAT3 inhibitor or a composition (e.g., pharmaceutical composition) comprising an immune checkpoint inhibitor may be adjusted with pharmaceutically acceptable acids, bases or buffers to enhance the stability or efficacy of the composition.

In some embodiments, the subject is administered a composition (e.g., pharmaceutical composition) comprising a STAT3 inhibitor orally. In some embodiments the composition (e.g., pharmaceutical composition) is orally administered in any orally acceptable dosage form including, but not limited to, liqui-gel tablets or capsules, syrups, emulsions and aqueous suspensions. Liqui-gels may include gelatins, plasticisers, and/or opacifiers, as needed to achieve a suitable consistency and may be coated with enteric coatings that are approved for use, e.g., shellacs. Additional thickening agents, for example gums, e.g., xanthum gum, starches, e.g., corn starch, or glutens may be added to achieve a desired consistency of the composition (e.g., pharmaceutical composition) when used as an oral dosage. If desired, certain sweetening and/or flavoring and/or coloring agents may be added.

In some embodiments, the subject is administered a composition (e.g., pharmaceutical composition) comprising a STAT3 inhibitor in a form suitable for oral administration such as a tablet, capsule, pill, powder, sustained release formulations, solution, and suspension. The composition (e.g., pharmaceutical composition) may be in unit dosage forms suitable for single administration of precise dosages. Pharmaceutical compositions may comprise, in addition to a compound as described herein a pharmaceutically acceptable carrier, and may optionally further comprise one or more pharmaceutically acceptable excipients, such as, for example, stabilizers, diluents, binders, and lubricants. In addition, the tablet may include other medicinal or pharmaceutical agents, carriers, and or adjuvants.

The dosage may vary depending upon the dosage form employed and the route of administration utilized. The exact formulation, route of administration and dosage can be chosen by the individual physician in view of the patient's condition. (See e.g., Fingl, et al., 1975, in the Pharmacological Basis of Therapeutics”). Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician. A course of therapy can comprise one or more separate administrations of a compound as described herein.

In some embodiments, the compound of Formula (I) is N-(1′,2-dihydroxy-1,2′-binaphthalen-4′-yl)-4-methoxybenzenesulfonamide, and N-(1′,2-dihydroxy-1,2′-binaphthalen-4′-yl)-4-methoxybenzenesulfonamide is administered to the subject as an oral dosage form comprising a composition comprising:
a. a therapeutically effective amount of N-(1′,2-dihydroxy-1,2′-binaphthalen-4′-yl)-4-methoxybenzenesulfonamide;
b. an emulsifier (e.g., glyceride), the emulsifier being present in the composition in a weight ratio of compound of Formula (I) to the emulsifier of about 1:1 to about 1:2 (e.g., about 1:1.5);
c. a solubilizer, the solubilizer being present in the composition in a weight ratio of compound of Formula (I) to solubilizer of about 1:1 to about 1:5 (e.g., about 1:2 to about 1:4, e.g., about 1:3);
d. a polyethylene glycol (PEG), the polyethylene glycol being present in the composition in a weight ratio of compound of Formula (I) to polyethylene glycol of about 1:2 to about 1:6 (e.g., about 1:3 to about 1:5, e.g., about 1:4);
e. a surfactant, the surfactant being present in the composition in a weight ratio of compound of Formula (I) to surfactant of about 2:1 to about 1:2 (e.g., about 1:1 to about 1:2, e.g. about 1:2); and
f. an antioxidant, the antioxidant being present in the composition in a weight ratio of compound of Formula (I) to antioxidant of about 10:1 to about 30:1 (e.g., about 15:1 to about 25:1, e.g., about 20:1).

In some embodiments, the emulsifier is present in the composition in a weight ratio of compound of Formula (I) to the emulsifier of about 1:1. In some embodiments, the emulsifier is present in the composition in a weight ratio of compound of Formula (I) to the emulsifier of about 1:2. In some embodiments, the emulsifier is present in the composition in a weight ratio of compound of Formula (I) to the emulsifier of about 1:3. In some embodiments, the emulsifier is present in the composition in a weight ratio of compound of Formula (I) to the emulsifier of about 1:4. In some embodiments, the emulsifier is present in the composition in a weight ratio of compound of Formula (I) to the emulsifier of about 1:5. In some embodiments, the emulsifier is present in the composition in a weight ratio of compound of Formula (I) to the emulsifier of about 1:6. In some embodiments, the emulsifier is present in the composition in a weight ratio of compound of Formula (I) to the emulsifier of about 1:7. In some embodiments, the emulsifier is present in the composition in a weight ratio of compound of Formula (I) to the emulsifier of about 1:1.5.

In some embodiments, the solubilizer is present in the composition in a weight ratio of compound of Formula (I) to solubilizer of about 1:1. In some embodiments, the solubilizer is present in the composition in a weight ratio of compound of Formula (I) to solubilizer of about 1:2. In some embodiments, the solubilizer is present in the composition in a weight ratio of compound of Formula (I) to solubilizer of about 1:3. In some embodiments, the solubilizer is present in the composition in a weight ratio of compound of Formula (I) to solubilizer of about 1:4. In some embodiments, the solubilizer is present in the composition in a weight ratio of compound of Formula (I) to solubilizer of about 1:5. In some embodiments, the solubilizer is present in the composition in a weight ratio of compound of Formula (I) to solubilizer of about 1:4. In some embodiments, the solubilizer is present in the composition in a weight ratio of compound of Formula (I) to solubilizer of about 1:6. In some embodiments, the solubilizer is present in the composition in a weight ratio of compound of Formula (I) to solubilizer of about 1:4. In some embodiments, the solubilizer is present in the composition in a weight ratio of compound of Formula (I) to solubilizer of about 1:7. In some embodiments, the solubilizer is present in the composition in a weight ratio of compound of Formula (I) to solubilizer of about 1:4. In some embodiments, the solubilizer is present in the composition in a weight ratio of compound of Formula (I) to solubilizer of about 1:8. In some embodiments, the solubilizer is present in the composition in a weight ratio of compound of Formula (I) to solubilizer of about 1:4. In some embodiments, the solubilizer is present in the composition in a weight ratio of compound of Formula (I) to solubilizer of about 1:9. In some embodiments, the solubilizer is present in the composition in a weight ratio of compound of Formula (I) to solubilizer of about 1:10. In some embodiments, the solubilizer is present in the composition in a weight ratio of compound of Formula (I) to solubilizer of about 1:11. In some embodiments, the solubilizer is present in the composition in a weight ratio of compound of Formula (I) to solubilizer of about 1:12. In some embodiments, the solubilizer is present in the composition in a weight ratio of compound of Formula (I) to solubilizer of about 1:13. In some embodiments, the solubilizer is present in the composition in a weight ratio of compound of Formula (I) to solubilizer of about 1:14. In some embodiments, the solubilizer is present in the composition in a weight ratio of compound of Formula (I) to solubilizer of about 1:15. In some embodiments, the solubilizer is present in the composition in a weight ratio of compound of Formula (I) to solubilizer of about 1:16. In some embodiments, the solubilizer is present in the composition in a weight ratio of compound of Formula (I) to solubilizer of about 1:17. In some embodiments, the solubilizer is present in the composition in a weight ratio of compound of Formula (I) to solubilizer of about 1:18. In some embodiments, the solubilizer is present in the composition in a weight ratio of compound of Formula (I) to solubilizer of about 1:19. In some embodiments, the solubilizer is present in the composition in a weight ratio of compound of Formula (I) to solubilizer of about 1:20.

In some embodiments, the polyethylene glycol is present in the composition in a weight ratio of compound of Formula (I) to polyethylene glycol of about 1:1. In some embodiments, the polyethylene glycol is present in the composition in a weight ratio of compound of Formula (I) to polyethylene glycol of about 1:2. In some embodiments, the polyethylene glycol is present in the composition in a weight ratio of compound of Formula (I) to polyethylene glycol of about 1:3. In some embodiments, the polyethylene glycol is present in the composition in a weight ratio of compound of Formula (I) to polyethylene glycol of about 1:4. In some embodiments, the polyethylene glycol is present in the composition in a weight ratio of compound of Formula (I) to polyethylene glycol of about 1:5. In some embodiments, the polyethylene glycol is present in the composition in a weight ratio of compound of Formula (I) to polyethylene glycol of about 1:6. In some embodiments, the polyethylene glycol is present in the composition in a weight ratio of compound of Formula (I) to polyethylene glycol of about 1:7. In some embodiments, the polyethylene glycol is present in the composition in a weight ratio of compound of Formula (I) to polyethylene glycol of about 1:8. In some embodiments, the polyethylene glycol is present in the composition in a weight ratio of compound of Formula (I) to polyethylene glycol of about 1:9. In some embodiments, the polyethylene glycol is present in the composition in a weight ratio of compound of Formula (I) to polyethylene glycol of about 1:10. In some embodiments, the polyethylene glycol is present in the composition in a weight ratio of compound of Formula (I) to polyethylene glycol of about 1:11. In some embodiments, the polyethylene glycol is present in the composition in a weight ratio of compound of Formula (I) to polyethylene glycol of about 1:12. In some embodiments, the polyethylene glycol is present in the composition in a weight ratio of compound of Formula (I) to polyethylene glycol of about 1:13. In some embodiments, the polyethylene glycol is present in the composition in a weight ratio of compound of Formula (I) to polyethylene glycol of about 1:14. In some embodiments, the polyethylene glycol is present in the composition in a weight ratio of compound of Formula (I) to polyethylene glycol of about 1:15. In some embodiments, the polyethylene glycol is present in the composition in a weight ratio of compound of Formula (I) to polyethylene glycol of about 1:16. In some embodiments, the polyethylene glycol is present in the composition in a weight ratio of compound of Formula (I) to polyethylene glycol of about 1:17. In some embodiments, the polyethylene glycol is present in the composition in a weight ratio of compound of Formula (I) to polyethylene glycol of about 1:18. In some embodiments, the polyethylene glycol is present in the composition in a weight ratio of compound of Formula (I) to polyethylene glycol of about 1:19. In some embodiments, the polyethylene glycol is present in the composition in a weight ratio of compound of Formula (I) to polyethylene glycol of about 1:20.

In some embodiments, the surfactant is present in the composition in a weight ratio of compound of Formula (I) to surfactant of about 4:1. In some embodiments, the surfactant is present in the composition in a weight ratio of compound of Formula (I) to surfactant of about 3:1. In some embodiments, the surfactant is present in the composition in a weight ratio of compound of Formula (I) to surfactant of about 2:1. In some embodiments, the surfactant is present in the composition in a weight ratio of compound of Formula (I) to surfactant of about 1:1. In some embodiments, the surfactant is present in the composition in a weight ratio of compound of Formula (I) to surfactant of about 1:2. In some embodiments, the surfactant is present in the composition in a weight ratio of compound of Formula (I) to surfactant of about 1:3. In some embodiments, the surfactant is present in the composition in a weight ratio of compound of Formula (I) to surfactant of about 1:4. In some embodiments, the surfactant is present in the composition in a weight ratio of compound of Formula (I) to surfactant of about 1:5. In some embodiments, the surfactant is present in the composition in a weight ratio of compound of Formula (I) to surfactant of about 1:6.

In some embodiments, the antioxidant is present in the composition in a weight ratio of compound of Formula (I) to antioxidant of about 2:1. In some embodiments, the antioxidant is present in the composition in a weight ratio of compound of Formula (I) to antioxidant of about 3:1. In some embodiments, the antioxidant is present in the composition in a weight ratio of compound of Formula (I) to antioxidant of about 4:1. In some embodiments, the antioxidant is present in the composition in a weight ratio of compound of Formula (I) to antioxidant of about 5:1. In some embodiments, the antioxidant is present in the composition in a weight ratio of compound of Formula (I) to antioxidant of about 6:1. In some embodiments, the antioxidant is present in the composition in a weight ratio of compound of Formula (I) to antioxidant of about 7:1. In some embodiments, the antioxidant is present in the composition in a weight ratio of compound of Formula (I) to antioxidant of about 8:1. In some embodiments, the antioxidant is present in the composition in a weight ratio of compound of Formula (I) to antioxidant of about 9:1. In some embodiments, the antioxidant is present in the composition in a weight ratio of compound of Formula (I) to antioxidant of about 10:1. In some embodiments, the antioxidant is present in the composition in a weight ratio of compound of Formula (I) to antioxidant of about 11:1. In some embodiments, the antioxidant is present in the composition in a weight ratio of compound of Formula (I) to antioxidant of about 12:1. In some embodiments, the antioxidant is present in the composition in a weight ratio of compound of Formula (I) to antioxidant of about 13:1. In some embodiments, the antioxidant is present in the composition in a weight ratio of compound of Formula (I) to antioxidant of about 14:1. In some embodiments, the antioxidant is present in the composition in a weight ratio of compound of Formula (I) to antioxidant of about 15:1. In some embodiments, the antioxidant is present in the composition in a weight ratio of compound of Formula (I) to antioxidant of about 16:1. In some embodiments, the antioxidant is present in the composition in a weight ratio of compound of Formula (I) to antioxidant of about 17:1. In some embodiments, the antioxidant is present in the composition in a weight ratio of compound of Formula (I) to antioxidant of about 18:1. In some embodiments, the antioxidant is present in the composition in a weight ratio of compound of Formula (I) to antioxidant of about 19:1. In some embodiments, the antioxidant is present in the composition in a weight ratio of compound of Formula (I) to antioxidant of about 20:1. In some embodiments, the antioxidant is present in the composition in a weight ratio of compound of Formula (I) to antioxidant of about 21:1. In some embodiments, the antioxidant is present in the composition in a weight ratio of compound of Formula (I) to antioxidant of about 22:1. In some embodiments, the antioxidant is present in the composition in a weight ratio of compound of Formula (I) to antioxidant of about 23:1. In some embodiments, the antioxidant is present in the composition in a weight ratio of compound of Formula (I) to antioxidant of about 24:1. In some embodiments, the antioxidant is present in the composition in a weight ratio of compound of Formula (I) to antioxidant of about 25:1. In some embodiments, the antioxidant is present in the composition in a weight ratio of compound of Formula (I) to antioxidant of about 26:1. In some embodiments, the antioxidant is present in the composition in a weight ratio of compound of Formula (I) to antioxidant of about 27:1. In some embodiments, the antioxidant is present in the composition in a weight ratio of compound of Formula (I) to antioxidant of about 28:1. In some embodiments, the antioxidant is present in the composition in a weight ratio of compound of Formula (I) to antioxidant of about 29:1. In some embodiments, the antioxidant is present in the composition in a weight ratio of compound of Formula (I) to antioxidant of about 30:1.

In some embodiments, the compound of Formula (I) is present in the formulation in a concentration of at least 50 mg/g (e.g., at least 60 mg/g, at least 70 mg/g, at least 80 mg/g, or at least 90 mg/g) (e.g., excluding the mass of a capsule shell). In some embodiments, the compound of Formula (I) is present in the formulation in a concentration of at least 50 mg/g. In some embodiments, the compound of Formula (I) is present in the formulation in a concentration of at least 60 mg/g. In some embodiments, the compound of Formula (I) is present in the formulation in a concentration of at least 70 mg/g. In some embodiments, the compound of Formula (I) is present in the formulation in a concentration of at least 80 mg/g. In some embodiments, the compound of Formula (I) is present in the formulation in a concentration of at least 90 mg/g.

In some embodiments, the emulsifier is a glyceride emulsifier. In some embodiments, the emulsifier comprises optionally polyglycolyzed medium- and/or long-chain mono-, di-, and/or tri-glyceride(s). In some embodiments, the emulsifier comprises a medium-chain mono-glyceride. In some embodiments, the emulsifier comprises a polyglycolyzed medium-chain mono-glyceride. In some embodiments, the emulsifier comprises a long-chain mono-glyceride. In some embodiments, the emulsifier comprises a polyglycolyzed long-chain mono-glyceride. In some embodiments, the emulsifier comprises a medium-chain di-glyceride. In some embodiments, the emulsifier comprises a polyglycolyzed medium-chain di-glyceride. In some embodiments, the emulsifier comprises a long-chain di-glyceride. In some embodiments, the emulsifier comprises a polyglycolyzed long-chain di-glyceride. In some embodiments, the emulsifier comprises a medium-chain tri-glyceride. In some embodiments, the emulsifier comprises a polyglycolyzed medium-chain tri-glyceride. In some embodiments, the emulsifier comprises a long-chain tri-glyceride. In some embodiments, the emulsifier comprises a polyglycolyzed long-chain tri-glyceride.

In some embodiments, the emulsifier is Labrasol®. In some embodiments, the emulsifier is Capmul® MCM. In some embodiments, the emulsifier is Capmul® MCM EP. In some embodiments, the emulsifier is Capmul® C8 EP. In some embodiments, the emulsifier is Capryol® 90.

In some embodiments, the solubilizer is a polyoxyl castor oil or a vitamin E polyethylene glycol succinate (TPGS). In some embodiments, the solubilizer is a polyoxyl castor oil. In some embodiments, the solubilizer is a vitamin E polyethylene glycol succinate. In some embodiments, the surfactant is Kolliphor® RH 40. In some embodiments, the solubilizer is Vitamin E TPGS.

In some embodiments, the polyethylene glycol (PEG) has an average molecular weight of about 200 to about 1000 (e.g., about 500 to about 700, or about 550 to about 650, or about 600). In some embodiments, the polyethylene glycol (PEG) has an average molecular weight of about 200 to 1000. In some embodiments, the polyethylene glycol (PEG) has an average molecular weight of about 500 to 700. In some embodiments, the polyethylene glycol (PEG) has an average molecular weight of about 550 to 650. In some embodiments, the polyethylene glycol (PEG) has an average molecular weight of about 600.

In some embodiments, the polyethylene glycol (PEG) is PEG200. In some embodiments, the polyethylene glycol (PEG) is PEG300. In some embodiments, the polyethylene glycol (PEG) is PEG400. In some embodiments, the polyethylene glycol (PEG) is PEG500. In some embodiments, the polyethylene glycol (PEG) is PEG600. In some embodiments, the polyethylene glycol (PEG) is PEG700. In some embodiments, the polyethylene glycol (PEG) is PEG800. In some embodiments, the polyethylene glycol (PEG) is PEG900. In some embodiments, the polyethylene glycol (PEG) is PEG1000.

In some embodiments, the surfactant is polysorbate (e.g., polysorbate 20). In some embodiments, the surfactant is polysorbate 20. In some embodiments, the surfactant is polysorbate 40. In some embodiments, the surfactant is polysorbate 60. In some embodiments, the surfactant is polysorbate 80.

In some embodiments, the antioxidant is vitamin E. In some embodiments, the antioxidant is ascorbyl palmitate. In some embodiments, the antioxidant is butylated hydroxytoluene. In some embodiments, the antioxidant is triethyl citrate. In some embodiments, the antioxidant is citric acid.

In some embodiments, the composition further comprises a co-solvent (e.g., Transcutol®). In some embodiments, the composition further comprises Transcutol® HP.

In some embodiments, the methods as described herein contemplate administering to the subject an oral dosage form comprising the pharmaceutical composition as described herein contained within a capsule.

### EXAMPLES

In the examples set forth below, following abbreviations are used: ROA=route of administration; i.p.=intraperitoneal(ly); p.o=oral(ly); BID=twice a day; BIW=twice a week; PBS=phosphate buffered saline; FBS=fetal bovine serum; N/A=not available or not applicable; SD=standard deviation; SEM=standard error of mean; TGI=tumor growth inhibition; TV=tumor volume.

### Example 1. In Vivo Efficacy Study and Rechallenge Study of TTI-101+Anti-PD-1 in the Treatment of Subcutaneous Liver Cancer Syngeneic Model (H22) in Female BALB/c Mice

The objective of efficacy study was to evaluate preclinically the in vivo therapeutic efficacy of TTI-101 alone and in combination with anti-PD-1 in the treatment of subcutaneous liver cancer syngeneic model H22 in female BALB/c mice. The objective of rechallenge study was to evaluate immune memory of the female BALB/c mice previously cured from subcutaneous liver cancer syngeneic model H22 by TTI-101 combined with anti-PD-1.

### Formulations

Dosing volume of Anti-PD-1 (clone RMP1-14) was adjusted for body weight. The concentration of anti-PD-1 was 1 mg/mL, and dosing volume was 10 μL/g. The anti-PD-1 dosing solution was prepared by diluting 1.372 mL of anti-PD-1 (8.2 mg/mL) with 9.878 mL of PBS.

Isotype control was made by diluting isotype control with PBS. Dosing volume was adjusted for body weight. The concentration of isotype control was 1 mg/mL, and dosing volume was 10 μL/g. For each preparation, 7.5 mL of dosing solution was prepared by diluting isotype control with PBS.

A composition of TTI-101 (72 mg/mL) dissolved in Kolliphor RH40, PEG600, Labrasol, Tween 20, and citric acid solution, was then dispersed in 1:2 water (vortexed) to form the dosing emulsion. The concentration of TTI-101 in the dosing emulsion was 24 mg/mL.

Vehicle formulation composed of Kolliphor RH40, PEG600, Labrasol, Tween 20, and citric acid solution, was then dispersed 1:2 in water (vortexed) to form the dosing emulsion.

### Cell Culture

The H22 tumor cells were maintained in vitro with RPMI1640 medium supplemented with 10% fetal bovine serum (FBS) at 37° C. in an atmosphere of 5% CO₂ in air. The cells in exponential growth phase were harvested and quantitated by cell counter before tumor inoculation.

### Tumor Inoculation

In efficacy study, each mouse was inoculated subcutaneously in the right front flank region with tumor cells (1×10⁶) in 0.1 ml of PBS for tumor development.

In rechallenge study, each mouse was inoculated subcutaneously in the left front flank region with tumor cells (1×10⁶) in 0.1 ml of PBS for tumor development. The day of inoculation was denoted as Day 0.

### Randomization

The randomization in efficacy study started when the mean tumor size reached approximately 73 mm³ (for groups 1-5) and 96 mm³ (for groups 6-9). Totally 82 mice were enrolled in the study and randomly allocated to 9 study groups, with 10 mice per group (groups 1-5) and 8 mice per group (groups 6-9). Randomization was performed based on “Matched distribution” method (Study Director TM software, version 3.1.399.19). The date of randomization was denoted as Day 1.

### Dosing

Each group was dosed according to the schedule shown in Table 1.

TABLE 1
Efficacy study design

| | | | Dose | Dosing | | Dosing |
|---|---|---|---|---|---|---|
| | | | Level | Volume | | Frequency& |
| Group | N | Treatment | (mg/kg) | (mL/kg) | ROA | Duration |
| 1 | 10 | Vehicle | 1.2 | 50 | p.o. | BID*3 weeks |
| | | | mg/mouse | (μL/mouse) | | |
| | | Isotype | 10 | 10 | i.p. | BIW*3 weeks |
| | | control | | | | |
| 2 | 10 | TTI-101 | 1.2 | 50 | p.o. | BID*3 weeks |
| | | | mg/mouse | (μL/mouse) | | |
| | | Isotype | 10 | 10 | i.p. | BIW*3 weeks |
| | | control | | | | |
| 3 | 10 | Vehicle | 1.2 | 50 | p.o. | BID*3 weeks |
| | | | mg/mouse | (μL/mouse) | | |
| | | Anti-PD-1 | 10 | 10 | i.p. | BIW*3 weeks |
| 4 | 10 | TTI-101 | 1.2 | 50 | p.o. | BID*3 weeks |
| | | | mg/mouse | (μL/mouse) | | |
| | | Anti-PD-1 | 10 | 10 | i.p. | BIW*3 weeks |
| 5 | 10 | Vehicle | 1.2 | 50 | p.o. | BID*5.5 days |
| | | | mg/mouse | (μL/mouse) | | |
| | | TTI-101 | 1.2 | 50 | p.o. | BID*15 days |
| | | | mg/mouse | (μL/mouse) | | |
| | | Anti-PD-1 | 10 | 10 | i.p. | BIW*3 weeks |

N: animal number per group;
BID meant 8 hours apart between two doses in one day;
For vehicle and TTI-101, only one dose was given on the day of randomization and termination.

Test articles were given between 8 am-9 am then again between 4 pm-5 pm.

For group 5, TTI-101 was initiated 5 days post anti-PD-1 dosing per the request.

In the rechallenge study, Groups 4, 5, and 10 were rechallenged according to Table 2.

TABLE 2
Rechallenge study design

| | | | | Cell |
|---|---|---|---|---|
| | | Tumor Inoculation | Mice | Inoculation |
| Group | N | Description | Description | Site |
| 4 | 3 | 1 × 10⁶ H22 tumor | Mice with | in the left front |
| | | cells in 0.1 mL PBS | complete tumor | flank region |
| | | | regression after | |
| | | | anti-PD-1 and TTI- | |
| | | | 101 (21 days) | |
| | | | treatment | |
| 5 | 2 | | Mice with | |
| | | | complete tumor | |
| | | | regression after | |
| | | | anti-PD-1 and TTI- | |
| | | | 101 (15 days) | |
| | | | treatment | |
| 10 | 7 | | Naïve mice | |

There was no treatment in the rechallenge study.

### Tumor Volume Measurement

Tumor volumes were measured twice per week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

### Endpoints

Tumor growth inhibition (TGI), T/C value and Survival was applied at the endpoint.

Tumor regression (REG)=[(T₀−Tᵢ)/T₀]×100%;

Ti as the mean tumor volume of the treatment group on the measurement day;

T₀ as the mean tumor volume of the treatment group on Day 0.

Tumor Growth Inhibition; TGI %=(1−Ti/Ci)*100;

Ti as the mean tumor volume of the treatment group on the measurement day;

Ci as the mean tumor volume of control group at the measurement day.

Treatment/Control (T/C) value (%) is an indicator of tumor response to treatment, and one of commonly used anti-tumor activity endpoint; T and C are the mean tumor volumes of the treatment and control groups, respectively, on a given day.

Tumor volume change_Treatment/Tumor volume change_Control (ΔT/ΔC)=(Tᵢ−T₀)/(Cᵢ−C₀)*100%; Tᵢ and Cᵢ as the mean tumor volumes of the treatment and control groups on the measurement day; T₀ and C₀ as the mean tumor volumes of the treatment and control groups on Day 0.

### Study Termination

The treatment has been performed for 21 days. Group 1 and group 2 were terminated on Day 21; group 3 was terminated on Day 51; group 4 and group 5 were extended for rechallenge study and was terminated on Day 130 (Day 24 in rechallenge study).

### Results

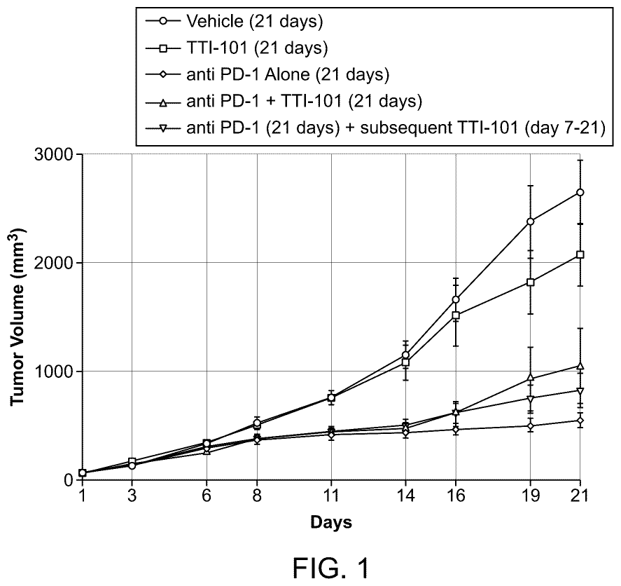

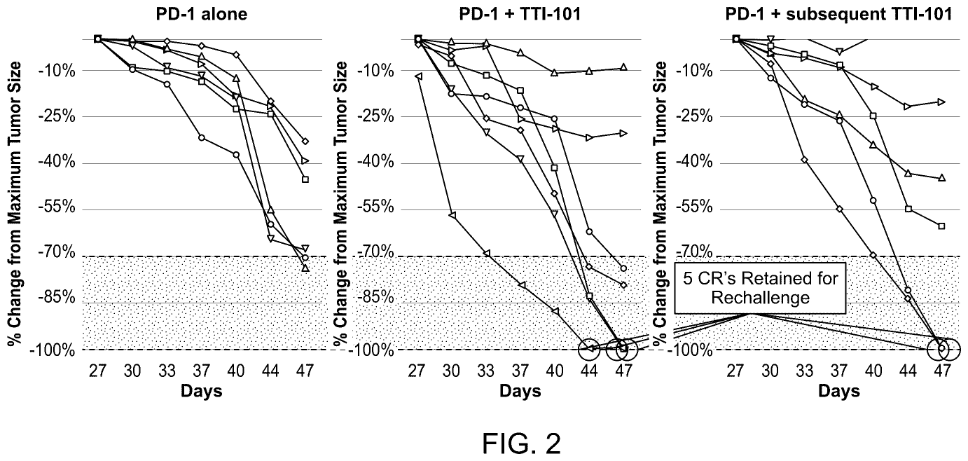

FIG. 1 and FIG. 2 show the in vivo efficacy of TTI-101±anti-PD-1 in H22 model through Day 21 and Day 47, respectively.

### Tumor Growth Inhibition (TGI)

The tumor growth inhibition in the treatment of subcutaneous liver cancer syngeneic model H22 is summarized in Table 3.

TABLE 3
Summary of tumor growth inhibition

| | | Tumor | T/C | TGI | | |
|---|---|---|---|---|---|---|
| | | volume | (%) | (%) | | |
| | | (mm³) ^{a} | on | on | | |
| | | (N) on | Day | Day | P | CR |
| Group | Treatment Description | Day 21 | 21 | 21 | value ^{b} | Ratio |
| 1 | Vehicle, 1.2 mg/mouse, 50 | 2647.11 ± | — | — | — | — |
| | μL/mouse, p.o., BID*3 weeks, | 293.71(10) | | | | |
| | Isotype control, 10 mg/kg, 10 | | | | | |
| | μL/g, i.p., BIW*3 weeks | | | | | |
| 2 | Isotype control, 10 mg/kg, 10 | 2073.36 ± | 78.33 | 21.67 | 0.743 | 0/10 |
| | μL/g, i.p., BIW*3 weeks, | 288.12(10) | | | | |
| | TTI-101, 1.2 mg/mouse, 50 | | | | | |
| | μL/mouse, p.o., BID*3 weeks | | | | | |
| 3 | Vehicle, 1.2 mg/mouse, 50 | 557.30 ± | 21.05 | 78.95 | 1.67E−06 | 0/10^{c} |
| | μL/mouse, p.o., BID*3 weeks, | 71.87(10) | | | | |
| | Anti-PD-1, 10 mg/kg, 10 μL/g, i.p., | | | | | |
| | BIW*3 weeks | | | | | |
| 4 | Anti-PD-1, 10 mg/kg, 10 μL/g, i.p., | 1050.55 ± | 39.69 | 60.31 | 8.11E−05 | 3/10 |
| | BIW*3 weeks, | 345.94(10) | | | | |
| | TTI-101, 1.2 mg/mouse, 50 | | | | | |
| | μL/mouse, p.o., BID*3 weeks | | | | | |
| 5 | Vehicle, 1.2 mg/mouse, 50 | 829.41 ± | 31.33 | 68.67 | 6.98E−05 | 3/10 |
| | μL/mouse, p.o., BID**5.5 days, | 158.26(10) | | | | |
| | Anti-PD-1, 10 mg/kg, 10 μL/g, i.p., | | | | | |
| | BIW* 3 weeks, | | | | | |
| | TTI-101, 1.2 mg/mouse, 50 | | | | | |
| | μL/mouse, p.o., BID*15 days | | | | | |
| | from day 7 | | | | | |

Note:
^{a}Mean ± SEM;
^{b} compared with group 1 tumor volume on Day 21 using Conover's non-parametric test, LOCF TV cutoff = 3000 mm³;
^{c}6 animals in group 3 displayed tumor regression on Day 51, while animals were terminated on Day 51 per the request of the sponsor. CR: tumor complete response, tumor regressed to 0.

In this study, the mean tumor size of the vehicle mice reached 2647 mm³ on Day 21 post randomization. TTI-101 administered at 1.2 mg/mouse, BID, displayed slight anti-tumor efficacy against H22 model, with TGI value of 21.67% on Day 21, however no statistically significant difference (P>0.05) was observed compared with vehicle group. Anti-PD-1 administered at 100 mg/kg, BIW, showed significant anti-tumor efficacy against H22 model, with TGI value of 78.95% on Day 21 (P<0.0001). 6 animals in group 3 displayed tumor regression on Day 51.

Co-administration of TTI-101 and anti-PD-1 produced significant anti-tumor efficacy against H22 model, with TGI value of 60.31% on Day 21, yielding a statistically significant difference of P<0.0001 when compared with vehicle group. 3 animals (3/10) in group 4 displayed complete tumor regression on Day 51 and remained tumor free until the study was terminated on Day 130.

Sequential dosing of TTI-101 7 days after anti-PD-1 produced significant anti-tumor efficacy against H22 model, with TGI value of 68.67% on Day 21, yielding a statistically significant difference of P<0.0001 when compared with vehicle group. 2 animals (2/10) in group 5 displayed complete tumor regression on Day 51 and remained tumor free until the study was terminated on Day 130.

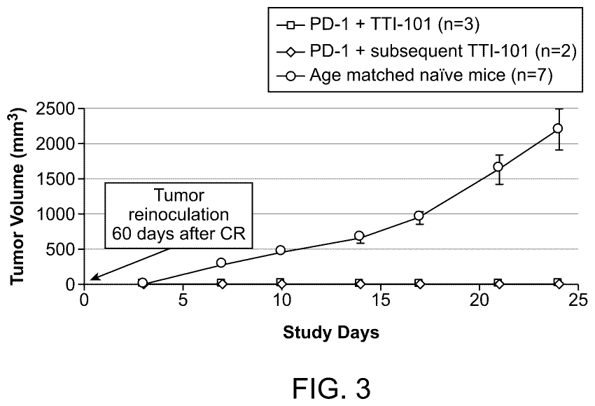

FIG. 3 shows that combination therapy of TTI-101 and anti-PD-1 resulting in tumor regression protects against tumor rechallenge. All naïve animals in group 10 developed tumors with 2 out of the 7 animals reaching tumor volume of 3000 mm³ 24 days post re-inoculation. Throughout the rechallenge study, 3 animals co-administrated of TTI-101 and anti-PD-1 (group 4) and 2 animals sequentially dosed of TTI-101 (started from Day 7) and anti-PD-1 (group 5) with complete tumor regression remained tumor free for both 1^{st} H22 tumor challenge in the right flank and for H22 rechallenge in the left flank until the study was terminated 24 days post re-inoculation.

### Example 2. Bioavailability and Pharmacokinetics of TTI-101 Following IP or PO Administration

Bioavailability and pharmacokinetics of TTI-101 in male Sprague-Dawley rats were evaluated following a single oral (PO) dose at 25 mg/kg (in labrasol:PEG400, 60:40% v/v) and a single intraperitoneal (IP) dose at 25.6 mg/kg (pH 7.4 PBS/propylene glycol/ethanol/DMSO, 20:70:5:5% v/v). Table 4 shows the exposure in rats dosed with a single administration of TTI-101. The results show that there was an unexpectedly 90% greater exposure by oral administration compared to interperitoneal administration.

TABLE 4
Area under curve (AUC) for PO vs. IP
administration of TTI-101 in rats

| | | |
|---|---|---|
| ROA | Dose | AUC |

| | | | |
|---|---|---|---|
| PO | 25 | mg/kg | 95,700 |
| IP | 25.6 | mg/kg | 50,300 |

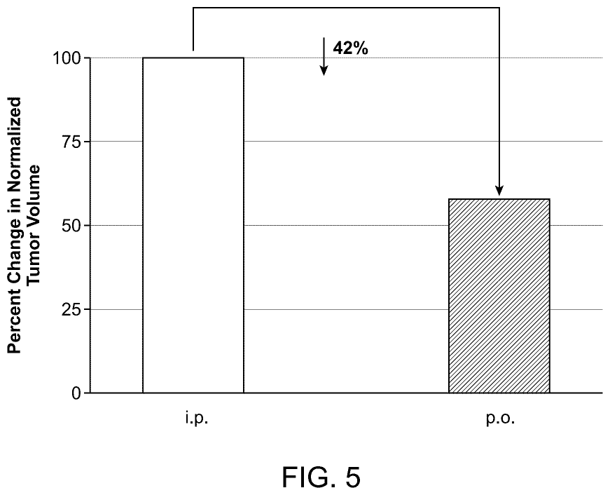

In addition, FIG. 5 shows the comparison of PO vs. IP administration in terms of its efficacy in cancer models such as murine H22 liver cancer model. Tumor volumes were measured at 18 days after twice daily dosing (50 mg/kg) of i.p. vs p.o. delivery in H22 mice (syngeneic liver cancer model) (n=8). Percent change in normalized tumor volume=average of TTI-101 treated mice/average of vehicle treated mice for both i.p. and p.o.

1. A compound of Formula (I): 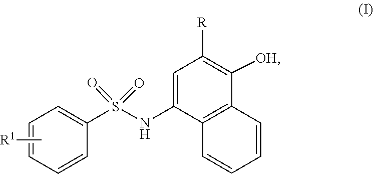
or a pharmaceutically acceptable salt thereof, wherein
R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl;
R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy;
and an immune checkpoint inhibitor, for use in treating cancer in a subject in need thereof, comprising:
a) orally administering to the subject an effective amount of the compound of Formula (I) or a pharmaceutically acceptable salt thereof, and
b) administering to the subject an effective amount of the immune checkpoint inhibitor.

2. The compound and immune checkpoint inhibitor for use of claim 1, wherein the compound of Formula (I) inhibits signal transducer and activator of transcription 3 (STAT3).

3. The compound and immune checkpoint inhibitor for use of claim 1, wherein the compound of Formula (I) inhibits signal transducer and activator of transcription 1 (STAT1).

4. The compound and immune checkpoint inhibitor for use of claim 1, wherein the compound of Formula (I) inhibits signal transducer and activator of transcription 5 (STAT5).

5. The compound and immune checkpoint inhibitor for use of claim 1, wherein the compound of Formula (I) is N-(1′,2-dihydroxy-1,2′-binaphthalen-4′-yl)-4-methoxybenzenesulfonamide, N-(3, 1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′] binaphthalenyl-4-yl)-benzenesulfonamide, or 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

6. The compound and immune checkpoint inhibitor for use of claim 5, wherein the compound of Formula (I) is N-(1′,2-dihydroxy-1,2′-binaphthalen-4′-yl)-4-methoxybenzenesulfonamide.

7. The compound and immune checkpoint inhibitor for use of claim 1, wherein the immune checkpoint inhibitor is a PD-1 inhibitor or a PD-L1 inhibitor.

8. The compound and immune checkpoint inhibitor for use of claim 7, wherein the PD-1 inhibitor is an anti-PD-1 antibody.

9. The compound and immune checkpoint inhibitor for use of claim 8, wherein the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717.

10. The compound and immune checkpoint inhibitor for use of claim 9, wherein the anti-PD-1 antibody is pembrolizumab.

11. The compound and immune checkpoint inhibitor for use of claim 7, wherein the PD-L1 inhibitor is an anti-PD-L1 antibody.

12. The compound and immune checkpoint inhibitor for use of claim 11, wherein the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

13. The compound and immune checkpoint inhibitor for use of any one of claims 1-12, wherein the immune checkpoint inhibitor is administered prior to administration of the compound of Formula (I).

14. The compound and immune checkpoint inhibitor for use of any one of claims 1-12, wherein the immune checkpoint inhibitor is administered concomitantly with administration of the compound of Formula (I).

15. The compound and immune checkpoint inhibitor for use of any one of claims 1-12, wherein the immune checkpoint inhibitor is administered subsequently to administration of the compound of Formula (I).

16. The compound and immune checkpoint inhibitor for use of any one of claims 1-15, wherein after three weeks or more of administration of the compound of Formula (I) and the immune checkpoint inhibitor, the subject has at least a partial remission.

17. The compound and immune checkpoint inhibitor for use of any one of claims 1-16, wherein after six weeks or more of administration of the compound of Formula (I) and the immune checkpoint inhibitor, the subject has complete remission.

18. The compound and immune checkpoint inhibitor for use of any one of claims 1-17, wherein the method reduces tumor size by at least 20% more within six weeks of administration relative to a subject administered immune checkpoint inhibitor alone.

19. A compound of Formula (I): 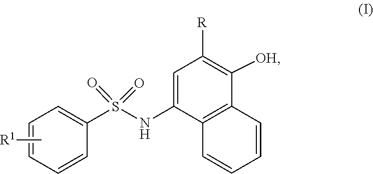
or a pharmaceutically acceptable salt thereof, wherein
R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl;
R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy;
and an immune checkpoint inhibitor, for use in a method of reducing the risk of recurrence of a cancer in a subject in need thereof, comprising:
a) orally administering to the subject an effective amount of the compound of Formula (I), or a pharmaceutically acceptable salt thereof, and
b) administering to the subject an effective amount of the immune checkpoint inhibitor.

20. A compound of Formula (I): 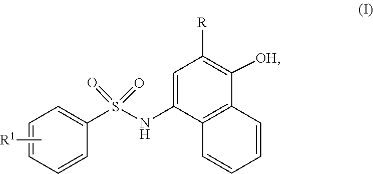
or a pharmaceutically acceptable salt thereof, wherein
R is selected from the group consisting of hydrogen, phenylsulfanyl, 2-hydroxy-naphthalen-1-yl, quinolin-8-ylsulfanyl, triazol-3-yl sulfanyl, and benzothiazol-2-ylsulfanyl;
R¹ is selected from the group consisting of hydrogen, methyl, chloro, bromo, methoxy, ethoxy, tert-butyl, nitro, methyl ester, acetamide, 1,4 dioxine, fluoro, trifluoro methoxy, acetyl, trifluoro methyl, propyl, cyclohexene, methoxy-phenoxy, chloro phenoxy, tolyloxy, and phenoxy;
and an immune checkpoint inhibitor, for use in a method of preventing recurrence of a cancer in a subject in need thereof, comprising:
a) orally administering to the subject an effective amount of the compound of Formula (I), or a pharmaceutically acceptable salt thereof, and
b) administering to the subject an effective amount of the immune checkpoint inhibitor.

21. The compound and immune checkpoint inhibitor for use of claim 19 or 20, wherein the immune checkpoint inhibitor is parenterally administered to the subject.

22. A compound of Formula (II): 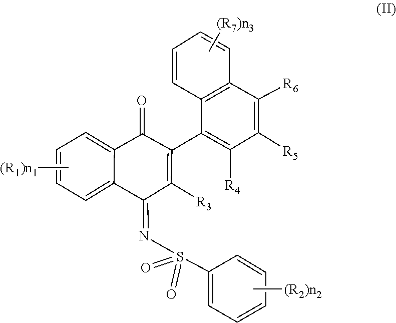
or a pharmaceutically acceptable salt thereof, wherein
each occurrence of R¹ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₁ is 0, 1, 2, 3, or 4;
each occurrence of R₂ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, cycloalkenyl, optionally substituted aryl, optionally substituted aryloxyl, or optionally substituted
n₂ is 0, 1, 2, 3, 4, or 5;
R₃ is hydrogen, halogen, cyano, nitro, CF₃, OCF₃, ORa, SRa, OC(═O)Rₐ, alkyl, alkenyl, cycloalkyl, or optionally substituted aryl or heteroaryl;
R₄ is hydrogen, halogen, cyano, nitro, CF₃, OCF₃, ORa, SRa, NRbRc, OC(═O)Ra, alkyl, alkenyl, or cycloalkyl;
each occurrence of R₅, R₆, and R₇ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₃ is 0, 1, 2, 3, or 4; and
each occurrence of Ra, Rb, and Rc is independently hydrogen, alkyl, cycloalkyl, alkenyl, cycloalkenyl, alkynyl, heterocycle, or aryl; or Rb and Rc together with the nitrogen atom to which they are bonded optionally form a heterocycle comprising 1-4 heteroatoms; and
an immune checkpoint inhibitor, for use in treating cancer in a subject in need thereof, comprising administering to the subject:
a) an effective amount of the compound of Formula (II), or a pharmaceutically acceptable salt thereof, and
b) an effective amount of the immune checkpoint inhibitor.

23. The compound and immune checkpoint inhibitor for use of claim 22, wherein the compound of Formula (II) inhibits signal transducer and activator of transcription 3 (STAT3).

24. The compound and immune checkpoint inhibitor for use of claim 22, wherein the compound of Formula (II) inhibits signal transducer and activator of transcription 1 (STAT1).

25. The compound and immune checkpoint inhibitor for use of claim 22, wherein the compound of Formula (II) inhibits signal transducer and activator of transcription 5 (STAT5).

26. The compound and immune checkpoint inhibitor for use of claim 22, wherein the compound of Formula (II) is a compound of Formula (III): 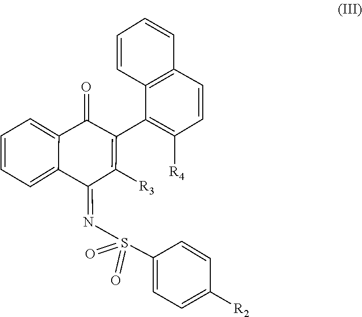
or a pharmaceutically acceptable salt thereof.

27. The compound and immune checkpoint inhibitor for use of claim 26, wherein the compound of Formula (III) is a compound of Formula (IV): 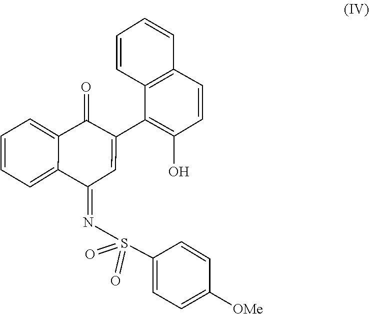 [SMILES: COc1ccc(S(=O)(=O)/N=C2\C=C(c3c(O)ccc4ccccc34)C(=O)c3ccccc32)cc1]
or a pharmaceutically acceptable salt thereof.

28. The compound and immune checkpoint inhibitor for use of any one of claims 22-27, wherein the immune checkpoint inhibitor is a PD-1 inhibitor or a PD-L1 inhibitor.

29. The compound and immune checkpoint inhibitor for use of claim 28, wherein the PD-1 inhibitor is an anti-PD-1 antibody.

30. The compound and immune checkpoint inhibitor for use of claim 29, wherein the anti-PD-1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK104, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717.

31. The compound and immune checkpoint inhibitor for use of claim 30, wherein the anti-PD-1 antibody is pembrolizumab.

32. The compound and immune checkpoint inhibitor for use of claim 28, wherein the PD-L1 inhibitor is an anti-PD-L1 antibody.

33. The compound and immune checkpoint inhibitor for use of claim 32, wherein the anti-PD-L1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-072.

34. The compound and immune checkpoint inhibitor for use of any one of claims 22-33, wherein the immune checkpoint inhibitor is administered prior to administration of the compound of Formula (II).

35. The compound and immune checkpoint inhibitor for use of any one of claims 22-33, wherein the immune checkpoint inhibitor is administered concomitantly with administration of the compound of Formula (II).

36. The compound and immune checkpoint inhibitor for use of any one of claims 22-33, wherein the immune checkpoint inhibitor is administered subsequently to administration of the compound of Formula (II).

37. The compound and immune checkpoint inhibitor for use of any one of claims 22-36, wherein the compound of Formula (II) is administered orally to the subject.

38. A compound of Formula (II): 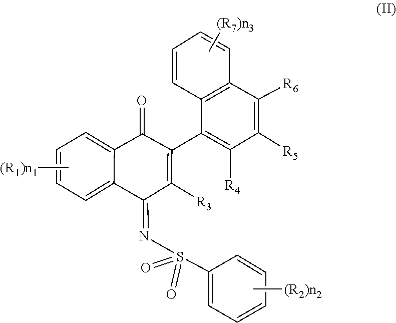
or a pharmaceutically acceptable salt thereof, wherein
each occurrence of R₁ is independently hydrogen, halogen, cyano, nitro, CF₃, OCF₃, ORa, SRa, C(═O)Ra, OC(═O)Ra, C(═O)ORₐ, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₁ is 0, 1, 2, 3, or 4;
each occurrence of R₂ is independently hydrogen, halogen, cyano, nitro, CF₃, OCF₃, ORa, SRa, C(═O)Ra, OC(═O)Ra, C(═O)ORₐ, NRbRc, NRbC(═O)Rc, C(═O)NRbRc, NRbC(═O)ORc, OC(═O)NRbRc, NRaC(═O)NRbRc, alkyl, alkenyl, cycloalkyl, cycloalkenyl, optionally substituted aryl, optionally substituted aryloxyl, or optionally substituted
n₂ is 0, 1, 2, 3, 4, or 5;
R₃ is hydrogen, halogen, cyano, nitro, CF₃, OCF₃, ORa, SRa, OC(═O)Ra, alkyl, alkenyl, cycloalkyl, or optionally substituted aryl or heteroaryl;
R₄ is hydrogen, halogen, cyano, nitro, CF₃, OCF₃, ORa, SRa, NRbRc, OC(═O)Ra, alkyl, alkenyl, or cycloalkyl;
each occurrence of R₅, R₆, and R₇ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₃ is 0, 1, 2, 3, or 4; and
each occurrence of Ra, Rb, and Rc is independently hydrogen, alkyl, cycloalkyl, alkenyl, cycloalkenyl, alkynyl, heterocycle, or aryl; or Rb and Rc together with the nitrogen atom to which they are bonded optionally form a heterocycle comprising 1-4 heteroatoms; and
an immune checkpoint inhibitor, for use in a method of reducing the risk of recurrence of a cancer in a subject in need thereof, comprising administering to the subject:
a) an effective amount of the compound of Formula (II), or a pharmaceutically acceptable salt thereof, and
b) an effective amount of the immune checkpoint inhibitor.

39. A compound of Formula (II): 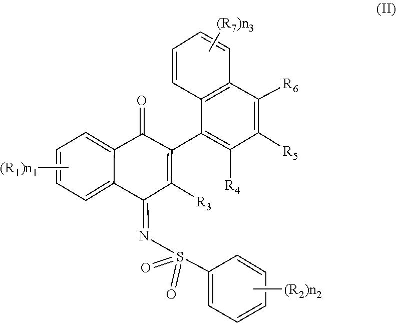
or a pharmaceutically acceptable salt thereof, wherein
each occurrence of R¹ is independently hydrogen, halogen, cyano, nitro, CF₃, OCF₃, ORa, SRa, C(═O)Ra, OC(═O)Ra, C(═O)ORₐ, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₁ is 0, 1, 2, 3, or 4;
each occurrence of R₂ is independently hydrogen, halogen, cyano, nitro, CF₃, OCF₃, ORa, SRa, C(═O)Ra, OC(═O)Ra, C(═O)ORₐ, NRbRc, NRbC(═O)Rc, C(═O)NRbRc, NRbC(═O)ORc, OC(═O)NRbRc, NRaC(═O)NRbRc, alkyl, alkenyl, cycloalkyl, cycloalkenyl, optionally substituted aryl, optionally substituted aryloxyl, or optionally substituted heterocycle;
n₂ is 0, 1, 2, 3, 4, or 5;
R₃ is hydrogen, halogen, cyano, nitro, CF₃, OCF₃, ORa, SRa, OC(═O)Ra, alkyl, alkenyl, cycloalkyl, or optionally substituted aryl or heteroaryl;
R₄ is hydrogen, halogen, cyano, nitro, CF₃, OCF₃, ORa, SRa, NRbRc, OC(═O)Ra, alkyl, alkenyl, or cycloalkyl;
each occurrence of R₅, R₆, and R₇ is independently hydrogen, halogen, cyano, nitro, CF₃, OCF₃, ORa, SRa, C(═O)Ra, OC(═O)Ra, C(═O)ORa, NRbRc, NRbC(═O)Rc, C(═O)NRbRc, NRbC(═O)ORc, OC(═O)NRbRc, NRaC(═O)NRbRc, alkyl, alkenyl, cycloalkyl, optionally substituted aryl, or optionally substituted heterocycle;
n₃ is 0, 1, 2, 3, or 4; and
each occurrence of Ra, Rb, and Rc is independently hydrogen, alkyl, cycloalkyl, alkenyl, cycloalkenyl, alkynyl, heterocycle, or aryl; or Rb and Rc together with the nitrogen atom to which they are bonded optionally form a heterocycle comprising 1-4 heteroatoms; and
an immune checkpoint inhibitor, for use in a method of preventing recurrence of a cancer in a subject in need thereof, comprising administering to the subject:
a) an effective amount of the compound of Formula (II), or a pharmaceutically acceptable salt thereof, and
b) an effective amount of the immune checkpoint inhibitor.

40. The compound and immune checkpoint inhibitor for use of any one of claims 1-39, wherein the immune checkpoint inhibitor is administered parenterally.

41. The compound and immune checkpoint inhibitor for use of any one of claims 1-40, wherein the cancer is head and neck cancer, lung cancer, liver cancer, breast cancer, ovarian cancer, colon cancer, multiple myeloma, prostate cancer, cervical cancer, brain cancer, pancreatic cancer, myelodysplastic syndrome, neuroblastoma, kidney cancer, or metastatic melanoma.

42. The compound and immune checkpoint inhibitor for use of claim 41, wherein the cancer is head and neck cancer, lung cancer, liver cancer, breast cancer, ovarian cancer, colon cancer, multiple myeloma, or pancreatic cancer.
